(19) United States
(12) Patent Application Publication
(10) Pub. No.: US 2026/0167715 A1
(43) Pub. Date: Jun. 18, 2026

(54) PD-1+CD38HICD8+ KILLER T CELLS AND USES THEREOF

(71) Applicant: GEORGETOWN UNIVERSITY, Washington, DC (US)

(72) Inventors: Pankaj N. GAUR, Bethesda, MD (US); Samir KHLEIF, Washington, DC (US); Seema GUPTA, Bethesda, MD (US); Vivek VERMA, Austin, MN (US)

(73) Assignee: GEORGETOWN UNIVERSITY, Washington, DC (US)

(21) Appl. No.: 19/124,454

(22) PCT Filed: Oct. 26, 2023
(86) PCT No.: PCT/US2023/077883

§ 371 Date: Apr. 25, 2025

Related U.S. Application Data
(60) Provisional application No. 63/380,989, filed on Oct. 26, 2022.

(51) Int. Cl. C07K 16/28; A61K 40/11

(52) U.S. Cl. CPC C07K 16/2803; A61K 40/11; C07K 2317/76

(57) ABSTRACT

Provided herein are methods of targeting PD-1⁺CD38^{hi}CD8⁺ T cells to reduce apoptosis of white blood cells (e.g., lymphocytes). Further provided are methods of inducing apoptosis of white blood cells in a subject by using adoptive cell therapy with PD-1⁺CD38^{hi}CD8⁺ T cells.

### PRIOR RELATED APPLICATION

This application claims the benefit of U.S. Provisional Application No. 63/380,989, filed on Oct. 26, 2022, which is hereby incorporated by reference in its entirety.

### BACKGROUND

PD-1⁺CD38^{hi}CD8⁺ T cells are highly relevant to immunotherapy resistance in cancer. Depleting these cells reverses resistance to immunotherapy by increasing the number of total and tumor antigen specific CD8⁺ T cells in the tumor microenvironment (TME), their activation and effector functions. However, the role of these cells in other pathologies remains unknown.

### SUMMARY

PD-1⁺CD38^{hi}CD8⁺ T cells are dysfunctional with an ability to kill target cells expressing CD31 receptor. These target cells include CD4 and CD8 T cells and endothelial cells. The killing mechanism is through the binding of CD38 on these cells to CD31 on target cells leading to degranulation of the high level of Granzyme B (GzmB) from the CD38⁺PD1⁺CD8⁺ T cells and induction of apoptosis in the target cells. Therefore, these cells were termed fratricidal CD8 T cell (T^{frat}). These cells are highly expressed in advanced COVID-19 patients (e.g., up to 30-60%), when normally they are less than 12% of total CD8 T cells). Also, they kill autologous CD4 and CD8 T cells of advanced COVID-19 patients that are known to be severely lymphocytopenic. Additionally, PD1⁺CD38^{hi}CD8⁺ T cells are induced as a result of bradykinin (BDK)-mediated immune signaling in a cAMP response element-binding protein (CREB-1)-dependent manner, leading to ARDS pathology in the lungs. Additionally, PD-1⁺CD38^{hi}CD8⁺ T cells are induced in autoimmune disease models in which infusion of these cells leads to reversal of symptoms.

Provided herein are methods of blocking CD38 binding to CD31 on white blood cells and endothelial cells. Methods of preventing degranulation of Gzm B in PD-1⁺CD38^{hi}CD8⁺ T cells are also provided. Further provided herein are methods of using PD-1⁺CD38^{hi}CD8⁺ T cells in adoptive cell therapy for the treatment of autoimmune diseases.

For example, provided herein is a method for decreasing apoptosis of white blood cells in a subject comprising decreasing binding of a CD38-expressing cell to a CD31-expressing white blood cell in the subject. In some methods, an agent that decreases binding of a CD38-expressing cell to a CD31-expressing white blood cell is administered to the subject. In some methods, a decrease in PD-1⁺CD38^{hi}CD8⁺ T cells, for example, by depleting PD-1⁺CD38^{hi}CD8⁺ T cells in the subject, reduced binding of PD-1⁺CD38^{hi}CD8⁺ T cells to CD31-expressing white blood cells or endothelial cells in the subject.

In some methods, the CD31-expressing white blood cell is selected from the group consisting of T lymphocytes, dendritic cells, natural killer cells and macrophages. In some methods, the CD31-expressing white blood cell is a T lymphocyte. In some methods, the T lymphocyte is a CD4+ T cell or a CD8+ T cell.

In some methods, the CD38-expressing cell is a CD8+ T cell. In some methods, the CD8+ T cell is a PD-1+CD38hiCD8+ T cell. In some methods, the agent is an antibody that specifically binds to CD31. In some methods, the agent is an antibody that specifically binds to CD38. In some methods, the agent reduces the level of PD-1+CD38hiCD8+ T cells in the subject. In some methods, degranulation of GzmB in PD-1+CD38hiCD8⁺ T cells is reduced in the subject. In some methods, transfer of GzmB from PD-1+CD38hiCD8⁺ T cells into target cells (for example, white blood cells, or endothelial cells) in the subject is reduced. In some methods, the subject has acute respiratory distress syndrome (ARDS) or an ARDS-associated infection (e.g., COVID-19).

Also provided is a method of treating an autoimmune disease in a subject comprising administering to the subject a population of PD-1+CD38hiCD8+ T cells.

Also provided is a method of treating an infection in a subject comprising administering to the subject a population of PD-1+CD38hiCD8+ T cells. Some methods further comprise producing the population of PD-1+CD38hiCD8+ T cells by contacting CD8+ T cells with a suboptimal antigen and/or a PD-1 inhibitor, prior to administering the population of PD-1+CD38hiCD8+ T cells to the subject.

In some methods, the CD8+ T cells are contacted with an agent that inhibits the interaction of PD-1 and its ligand(s) to produce a population of PD-1+CD38hiCD8+ T cells. In some methods, the population of PD-1+CD38hiCD8⁺ T cells is expanded prior to administration to the subject.

Any of the methods provided herein can further comprise administering a second therapeutic agent to the subject. In some methods, the second therapeutic agent is an immunomodulator. In some methods, the immunomodulator is an immunosuppressant or an immunostimulant.

The details of one or more embodiments are set forth in the description below. Other features, objects, and advantages will be apparent from the description and from the claims.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIGS. 1a-j show differential regulation of a CD8 T cell subtype expressing PD1 and CD38. (a) Percentage and mean fluorescence intensity (MFI) of IFN-γ, CD40L⁺, and CD69⁺PD1⁺CD38^{hi}, effector T cells (T_{eff}) and CD38 KD PD1⁺CD38^{hi}CD8 T cells determined by flow cytometry. Representative data from one of two experiments are shown. The error bars indicate the s.e.m. Statistical analysis was performed by one-way ANOVA (*P≤0.05, **P≤0.01, ***P≤0.001, ****P≤0.0001). Scrambled RNA (scRNA) was used a control for CD38 siRNA. b,c, Recall response in PD1⁺CD38^{hi} cells rechallenged with Ova-V with and without CD38 KD (b) as measured by percentage of IFN-γ, CD40L⁺, and CD69⁺ cells (c). For comparison, recall response in T_{eff} cells rechallenged with Ova is shown by the percentage of IFN-γ cells (c). d,e, Level of pro- and anti-inflammatory cytokines, and cytolytic molecules in PD1⁺CD38^{hi} and T_{eff} CD8 T cells generated in vitro (d); or isolated from untreated TC-1 tumors in vivo (e) in mice as determined by flow cytometry. Representative data from one of two experiments are shown. The error bars indicate the s.e.m. Statistical analysis was performed by unpaired, one-tailed Student's t-test (*P≤0.05, **P≤0.01, ***P≤0.001, ****P≤0.0001). f-j, RNA and ATAC-seq analysis in PD-1⁺CD38^{hi} and T_{eff} cells generated from OT1 CD8⁺ T cells. (f) Principal Component Analysis (PCA) of RNA-seq analysis in IL-2-treated, PD-1⁺CD38 h and T_{eff} CD8 T cells. (g) Venn diagram (left) showing the pairwise comparisons for upregulated and downregulated gene analysis in IL-2-treated, PD-1⁺CD38^{hi} and T_{eff} CD8 T cells and GO pathway enrichment analysis, utilizing the differentially expressed upregulated and downregulated genes in PD-1⁺CD38^{hi} and T_{eff} CD8 T cells (right) (RNA-seq analysis). (h) The top 50 differentially expressed genes (DEGs) associated with the principal component 2 (PC2) axis in IL-2-treated, PD-1⁺CD38^{hi} and T_{eff} CD8 T cells by RNA-seq analysis. (i) Heat map analysis of inhibitory and inflammatory cytokine genes in PD-1⁺CD38^{hi} and T_{eff} CD8 T cells as determined by RNA-seq analysis. (j) PD-1⁺CD38^{hi} T cells have less promoter accessibility: ATAC-seq Tn5 nick site density in T_{eff} and PD1⁺CD38^{hi} T cell promoters. Log ratio (“minus”) vs average expression (“MA”) plot showing relative accessibility of macs2 peaks. Red indicates differential peak accessibility at an FDR<0.05 (top). Heatmap showing row-scaled promoter accessibility for promoter-restricted peaks that are differentially regulated at an FDR<0.05, |log 2 FC|>1.2 (bottom). Pathway terms with FDR<0.05 and the genes associated with these terms are depicted at right.

FIGS. 2a-g show the metabolic characteristics of PD1⁺CD38^{hi} CD8 T cells. a, b, PCA plots, volcano plots, and heat maps for different metabolites (a) and lipids (b) in PD-1⁺CD38^{hi} and T_{eff} CD8 T cells. c-d, Number of CD8⁺ T cells positive for NBDG (glucose uptake rates) and BODIPY (FA uptake) (c), TMRML and TMRM^{h}(mitochondrial potential) and MitoFM (mitochondrial mass) (d) with respective expression levels (MFI) in PD-1⁺CD38^{hi} cells compared to T_{eff} CD8 T cells. Representative results from one of the two experiments performed in triplicate are shown. The error bars indicate the s.e.m. Statistical analysis was performed by unpaired, one-tailed Student's t-test (*P≤0.05, **P≤0.01, ***P≤0.001, ****P≤0.0001). (e) larger mitochondria with disorganized cristae were found as indicated by a lower cristae density, number and score (f) DCFDA expression, showing ROS levels in PD-1⁺CD38^{hi} cells compared to T_{eff} cells. (g) Metabolic characteristics of PD-1⁺CD38^{hi} and T_{eff} CD8⁺ T cells, showing oxygen consumption rate (OCR, basal and maximal), spare respiration capacity (SRC) and extracellular acidification rates (ECAR). Representative results from one of the two experiments performed in triplicate are shown. The error bars indicate the s.e.m. Statistical analysis was performed by unpaired, one-tailed Student's t-test (*P K 0.05, ****P≤0.0001).

FIGS. 3a-j show that PD-1⁺CD38^{hi} CD8 T cells kill T cells in a contact-dependent manner mediated by CD38:CD31 interaction. a-c, Scheme for apoptosis assay to check the ability of PD-1⁺CD38^{hi} cells to induce apoptosis and loss of viability in target pMel-1 CD8 T cells in a contact-dependent or independent manner using trans-well chamber assay (a), FACS micrograph and statistical analysis of the frequency of the annexin V-positive (b) and annexin-V-negative live (c) target cells when incubated in contact or separated (M: membrane) from the PD-1⁺CD38^{hi} cells. Representative data from one of two experiments are shown. The error bars indicate the s.e.m. Statistical analysis was performed by one-way ANOVA (****P≤0.0001, ns, not significant). (d) Target CD8 T cells obtained from the spleens of pMel-1 mice, and target CD8 and CD4 T cells obtained from the spleens of C57BL/6 mice were incubated with PD1⁺CD38^{hi} CD8 T cells and number of live target cells was evaluated by a viability assay (live/dead cell staining). e-g, Schematic of the adoptive transfer experiment and the gating strategy (e), apoptosis levels (annexin V MFI) and number of live cells in adoptively transferred target CD8 (f) and CD4 (g) T cells isolated from the spleen of Rag1^{-/-} mice that were inoculated with PD-1⁺CD38^{hi} cells one day after transfer of target cells. Representative data from one of two experiments are shown. The error bars indicate the s.e.m. Statistical analysis was performed by unpaired, one-tailed Student's t-test (*P≤0.05, **P≤0.01). (h) Schematic for isolation of PD-1⁺CD38^{hi} and CD8⁺CD38^{lo} cells to check the involvement of CD38:CD31 interaction in inducing apoptosis in target pMel-1 CD8 T cells. In separate groups either CD38 or CD31 was knocked down using specific siRNA and also anti-CD38 or anti-CD31 was used to block the interaction of CD38 and CD31. (i) Annexin V levels or numbers in target CD8 cells when incubated with T^{frat} killer cells in the presence or absence of anti-CD38 or CD38KD. (j) Annexin V levels or numbers in target CD8 T cells in the presence of absence of anti-CD31 or CD31KD CD8⁺ target cells. Representative data from one of two experiments are shown. The error bars indicate the s.e.m. Statistical analysis was performed by one-way ANOVA (*P≤0.05, **P≤0.01, ***P≤0.001, ****P≤0.0001, ns, not significant.

FIGS. 4a-m show that T^{frat} cells kill by transferring Granzyme B (Gzm B) into target cells and CD38:CD31 interaction induces Gzm B degranulation in killer cells through Zap70-PI3K-RAC-ERK pathway. (a) Expression of annexin-V in mouse target CD8 T cells after incubation with killer T^{frat} cells in the presence or absence of Gzm B inhibitor. (b) Schematic for the generation of killer PD-1⁺CD38^{hi} cells and to check their ability to transfer Gzm B into target cells. c-d, Expression and frequency of Gzm B⁺ in target CD8 (c) and CD4 (d) T cells after co-incubation with killer (T^{frat}) cells. e-f, Degranulation in T^{frat} cells was estimated by CD107a expression (e) and MFI of Gzm B in target CD8 cells (f) after the two populations were incubated in the presence or absence of anti-CD31 or anti-CD38. Representative data from one of two experiments are shown. The error bars indicate the s.e.m. Statistical analysis was performed by one-way ANOVA (**P≤0.01, ***P≤0.001, ****P≤0.0001). (g) Schematic of determination of transfer of Gzm B from killer T^{frat} cells into target cells by immunofluorescence analysis. (h) Target (no nuclear staining) and T^{frat} (with nuclear staining) CD8 T cells stained for Gzm B. (i-j) Estimation of transfer of Gzm B from killer T^{frat} (blue) into target (non-labelled in ‘bright field’) CD8 T cells by immunofluorescence analysis at various time points (5 min to 300 min); ‘Merged’ is to show Gzm B on dark field for better visualization (i) and j shows the images at larger magnification for 180 min. (k) Estimation of various signaling molecules implicated in granule movement in PD-1⁺CD38^{hi} cells treated with recombinant (r) CD31 by Western blot analysis. 3-actin is shown as an internal control. On the right, densitometry analysis of various signaling molecules estimated is shown. (1) Degranulation in killer T^{fat} cells following incubation with rCD31, and PI3K and ERK inhibition by flow cytometry. Representative data from one of two experiments are shown. The error bars indicate the s.e.m. Statistical analysis was performed by one-way ANOVA (*P≤0.05, **P≤0.01, ***P≤0.001, ****P≤0.0001). (m) Overview of signaling pathways mediated by CD38 and CD31 implicated in Gzm B production and granule movement in cells.

FIGS. 5a-g show that CD38 depletes NAD leading to SIRT1-FOXO1-TCF7-mediated increased Gzm B production in killer T^{frat} cells. (a) Frequency and expression of Gzm B in T^{frat} with and without CD38KD compared to T_{eff} cells as determined by flow cytometry. (b) NAD levels in T^{frat} and T_{eff} cells. (c) SIRT1 activity by fluorometric analysis (left) and expression by Western blot analysis (right; expression of tubulin is shown as a control) in T^{frat} and T_{eff} cells. d-e, Expression of FOXO1 and TCF7 in T^{frat} and T_{eff} cells at protein (by Western blot analysis; Lamin B is shown as a control) (d) and RNA (by qRT-PCR analysis) (e) level. Numbers on the blots show the relative expression after densitometric analysis. Representative data from one of two experiments are shown. The error bars indicate the s.e.m. Statistical analysis was performed by unpaired, one-tailed Student's t-test (*P≤0.05, **P≤0.01, ***P≤0.001, ****P≤0.0001). f-g, Gzm B levels after the repletion of NAD (f) or addition of SIRT1 activator (g) in T^{frat} compared to T_{eff} CD8⁺ T cells. Representative data from one of two experiments are shown. The error bars indicate the s.e.m. Statistical analysis was performed by one-way ANOVA. (*P K 0.05, **P≤0.01, ***P≤0.001).

FIGS. 6a-n show that PD-1⁺CD38^{hi} CD8 cells suppress autoimmune disease. a, Number of PD1⁺CD38^{hi} CD8⁺ T cells in the spleen of experimental autoimmune encephalomyelitis (EAE) mice compared to the WT mice. (b) Frequency and level of Gzm B⁺PD1⁺CD38^{hi} and T_{eff} CD8⁺ T cells in the spleen of EAE mice. (c) In vitro killing of pMel-1 target cells by PD1⁺CD38^{hi} CD8⁺ T cells obtained from the EAE mice with or without anti-CD38 compared with the killing induced by T_{eff} cells. d-f, Number of PD1⁺CD38^{hi} CD8⁺ T cells in two cohorts of SLE patients (d-e) and MS patients (f) compared with healthy individuals. Frequency of Gzm B⁺PD1⁺CD38^{hi} and T_{eff} CD8⁺ T cells in SLE (g) and MS (h) patients. (i) Ability of Tra^{t} cells compared with T_{eff} cells both isolated from SLE patients to kill autologous target CD4 T cells in the presence or absence of anti-CD38 as determined by annexin V staining and viability assay by flow cytometry. Each symbol represents an independent patient. The error bars indicate the s.e.m. Statistical analysis was performed by unpaired, one-tailed Student's t-test (*P K 0.05, **P K 0.01, ns, not significant). (j) Schematic of induction of EAE and infusion of PD1⁺CD38^{hi} CD8⁺ T cells and grading for clinical scores. (k) EAE clinical score and mouse survival. (1-n) Analysis of immune response in various tissues (spleen, brain and lymph nodes, LN) from untreated EAE mice or EAE mice infused with T^{frat} cells by flow cytometry. The error bars indicate the s.e.m. Statistical analysis was performed by unpaired, one-tailed Student's t-test. Survival in the two groups was compared using log-rank (Mantel-Cox) tests (*P≤0.05, **P≤0.01, ***P≤0.001).

FIGS. 7a-e show that PD-1⁺CD38^{hi} CD8⁺ T cells from advanced COVID-19 patients kill T cells. (a) Frequency of T^{frat} cells in the blood of healthy individuals (control), convalescent, and severe COVID-19 patients. (b) Frequency of T^{f}t cells in the bronchoalveolar lavage fluid (BALF) of COVID-19 patients with severe/critical (S/C) disease compared to that from moderate (O) disease and healthy controls (HC) (analysis was performed using publicly available single-cell RNA-seq data). Each symbol corresponds to one individual. The error bars indicate the s.e.m. Statistical analysis was performed by one-way ANOVA (*P≤0.05, **P≤0.01). d, MFI of Gzm B in T^{frat} and T_{eff} cells from the peripheral blood of severe COVID-19 patients estimated by flow cytometry. (e) Ability of T^{frat} compared with T_{eff} isolated from severe COVID-19 patients to kill autologous CD4 T cells as determined by annexin-V staining by flow cytometry. Each line represents an independent patient. The error bars indicate the s.e.m. Statistical analysis was performed by unpaired, one-tailed Student's t-test (*P≤0.05, **P≤0.01, ns, not significant).

FIGS. 8a-g show a. The gating strategy in vitro. b. Percentage of IFN-γ, CD40L⁺, and CD69⁺ IL-2-treated, PD1⁺CD38^{h}, and T_{eff} CD8 T cells determined by flow cytometry. Representative data from one of two experiments are shown. The error bars indicate the s.e.m. Statistical analysis was performed by one-way ANOVA (***P≤0.001, ****P≤0.0001). c. The gating strategy in vivo. (d) Volcano plots showing DEGs between IL-treated, PD1⁺CD38^{hi} and T_{eff} CD8 T cells by RNA-seq analysis. e. A heatmap showing the global top 50 DEGs IL-treated, PD1⁺CD38^{hi} and T_{eff} CD8 T cells by RNA-seq analysis. (f) Exhaustion and effector like DEGs in IL-2-treated, PD-1⁺CD38^{hi} and T_{eff} CD8 T cells by RNA-seq analysis. (g) Volcano plot illustrates differentially open promoters with |log 2 FC|>1.2 by ATAC-seq analysis.

FIGS. 9a-c show the results of apoptosis experiments. (a-c) Induction of apoptosis in target CD8 T cells after incubation with PD1⁺CD38^{hi} or T_{eff} CD8⁺ T cells by caspase3/7 staining (a) and annexin V staining with or without anti-CD38 (b) or CD31KD in target cells (c).

FIGS. 10a-f show results using a mouse model of EAE and SLE and patients with SLE and COVID-19: (a) Gating strategy in splenocytes from EAE and WT mice. b. Percentage of PD1⁺CD38 cells in CD8 T cells was analyzed in the spleen of SLE mice with severe disease (Sle1Tg7 and Sle1TLR9KO) compared to the mice with mild SLE disease (Sle1) or TLR7KO (Sle1TLR7KO) at 9 months. Error bars: SEM. (*P≤0.05, **P≤0.01). c. Gating strategy for cells from patients with SLE. (d) Ability of T^{frat} cells compared with T_{eff} cells both isolated from SLE patients to kill autologous target CD4 T cells in the presence or absence of anti-CD38 as determined by annexin V staining and viability assay by flow cytometry. Each line represents an independent patient. The error bars indicate the s.e.m. Statistical analysis was performed by unpaired, one-tailed Student's t-test (*P≤0.05). e. Gating strategy for endogenous and infused cells in EAE mice. e. Schematic of induction of EAE and infusion of induced regulatory T cells (iTregs) is shown on the top. EAE clinical score and mouse survival is shown at the bottom. The error bars indicate the s.e.m. For EAE score, statistical analysis was performed by unpaired, one-tailed Student's t-test. Survival in the two groups was compared using log-rank (Mantel-Cox) tests (**P≤0.01, ****P≤0.0001).

FIG. 11A-E shows that bradykinin (BK) induces PD1⁺CD38^{hi}CD8⁺ cells in a CREB-1 dependent manner. (A) Frequency of PD1⁺CD38^{hi}CD8⁺ cells induced after BK treatment at various concentrations of normal-human CD8 T cells and mouse CD8 T cells determined by flow cytometry. (B) BK induces PD1⁺CD38^{hi}CD8⁺ cells by activating CREB in CD8 T cells. (C) CD38 depletion reduces number of BK-inducted PD1⁺CD38^{hi}CD8⁺ T cells in the lungs of wild-type mice. (E) Number of PD1⁺CD38^{hi} CD8⁺ T cells was accompanied with a severe lung pathology in BK-treated mice, similar to the ARDS features.

FIGS. 12A-H show that PD1⁺CD38^{hi}CD8⁺ cells induce killing in dendritic cells (DCs), Macrophages, natural killer (NK) cells and endothelial cells (ECs). (A-B) Scheme for estimation of killing in DCs, macrophages and NK cells from spleen of WT mice that were treated with intravenous infusion of PD1⁺CD38^{hi}CD8⁺ cells (A) and gating strategy for various cell populations (B).

(C-E) Estimation of frequencies and respective annexin expression in DCs (C) macrophages (D) and NK cells (E) after treatment with PD1⁺CD38^{hi}CD8⁺ cells as described in A. (F-G) Frequency of CD31⁺ cells in live C166 endothelial cells (F) and their killing by PD1⁺CD38^{hi} and PD1⁺CD38^{lo} (T_{eff}) cells as estimated by caspase3/7 staining by flow cytometry (G). Representative data from one of two experiments are shown. The error bars indicate the s.e.m. (H) Ability of PD1⁺CD38^{hi}CD8⁺ cells isolated from severe COVID-19 patients to kill HUVEC endothelial cells as determined by annexin-V staining by flow cytometry. The error bars indicate the s.e.m. Statistical analysis was performed by unpaired, one-tailed Student's t-test. *P≤0.05.

### DETAILED DESCRIPTION

PD1⁺CD38^{hi}CD8⁺ T cells, phenotypically and functionally, form a distinct subtype of CD8 Tcells with a novel function. These cells kill target cells expressing CD31 receptor. This includes CD4 and CD8 T cells and endothelial cells. PD1⁺CD38^{hi}CD8⁺ T cells kill cells through the binding of CD38 on these cells to CD31 on target cells leading to degranulation of Gzm B in the CD38⁺PD1⁺CD8 T cells, its transfer into the target cells, and induction of apoptosis in the target cells. These cells are highly expressed in advanced COVID-19 that are known to be severely lymphocytopenic. Therefore, targeting CD38:CD31 interactions, for example, the interaction between PD1⁺CD38^{hi}CD8⁺ T cells and CD31-expressing cells in a subject is useful for treating acute respiratory distress syndrome (ARDS) and ARDS-associated infections such as, for example, COVID-19.

As described herein, PD1⁺CD38^{hi}CD8⁺ T cells produce high levels of both pro- and anti-inflammatory cytokines. These cells co-express both effector and exhaustion associated genes. Additionally, they have a closed chromatin structure and yet high expression of genes for effector cytokines. The cells demonstrate metabolic catastrophe identified by upregulated metabolites and lipids. The cells also show a higher mitochondrial mass with leaky mitochondria, showing high reactive oxygen species (ROS) production. Additionally, PD1⁺CD38^{hi}CD8⁺ T cells have indiscriminate contact dependent cytotoxicity mediated through a CD38:CD31 interaction and (Gzm B) transfer to the target cells (CD8, CD4, dendritic cells, natural killer cells, endothelial cells, macrophages), leading to lymphocytopenia.

Furthermore, these cells are induced in autoimmune disease models, such as, for example, the experimental autoimmune encephalomyelitis (EAE) and SLE mouse models, and infusion of these cells into mice with EAE leads to rescue of the mice and reversal of symptoms. Higher numbers of these cells were found in patients with autoimmune diseases also. PD1⁺CD38^{hi}CD8⁺ T cells obtained from the SLE patients showed an ability to kill autologous target CD4⁺ T cells. Since PD1⁺CD38^{hi}CD8⁺ T cells have immune protective effects, these cells are useful for treating autoimmune disorders.

PD-1⁺CD38^{hi}CD8⁺ T Cells

PD-1⁺CD38^{hi}CD8⁺ T cells are dysfunctional T cells that can be targeted to treat inflammation-induced respiratory disorders, for example, COVID-19, ARDS and other ARDS-associated infections (e.g., viral infections). PD-1⁺CD38^{hi}CD8⁺ T cells can also be used to treat an autoimmune disorders or infection in a subject, by increasing the level of PD-1⁺CD38^{hi}CD8⁺ T cells in a subject, for example, by administering a population of PD-1⁺CD38^{hi}CD8⁺ T cells to the subject or by inducing production of PD-1⁺CD38^{hi}CD8⁺ T cells in the subject As used herein, dysfunctional T cells are T cells that do not react to repeated immune stimulation and/or fail to generate immune memory.

As described herein, PD-1⁺CD38^{hi}CD8⁺ T cells phenotypically and functionally form a distinct subtype of CD8 T cells with the ability to kill target cells expressing the CD31 receptor. Cell-killing or cytotoxicity is mediated through binding of CD38 to CD31 on target cells, for example, white blood cells or endothelial cells. When CD38⁺ T cells bind to CD31-expressing target cells, for example white blood cells, (Gzm B) is transferred from the CD38⁺ T cells (e.g., PD-1⁺CD38^{hi} CD8⁺ T cells), to the target cells to induce apoptosis in the target cells. Therefore, the PD-1⁺CD38^{hi}CD8⁺ T cells can be targeted, for example, by contacting the cells with an agent that decreases or inhibits binding of CD38 to CD31, for example, to decrease or inhibit binding between CD38 on the PD-1⁺CD38^{hi}CD8⁺ T cells and CD31 on the target cell. In addition, the number of PD-1⁺CD38^{hi}CD8⁺ T cells can also be reduced in the subject, for example, by depleting PD-1⁺CD38^{hi}CD8⁺ T cells in the subject. Decreasing the CD38:CD31 interaction between PD-1⁺CD38^{hi} CD8⁺ T cells and CD31-expressing cells and/or decreasing the number of PD-1⁺CD38^{hi}CD8⁺ T cells is useful for treating diseases associated with respiratory inflammation or distress.

It was also discovered that increasing the number of PD-1⁺CD38^{hi}CD8⁺ T cells in a subject, for example, by administering a population of PD-1⁺CD38^{hi}CD8⁺ T cells to the subject or inducing production of PD-1⁺CD38^{hi}CD8⁺ T cells, is useful for treating autoimmune disorders.

Methods for producing PD-1⁺CD38^{hi}CD8⁺ T cells are known in the art. Antigens/peptides that suboptimally prime/activate T cells through their T cell receptors (TCRs), or blockade of PD-1 in CD8⁺ T cells before antigenic stimulation (both suboptimal and optimal antigens) can be used for induction of PD1⁺CD38^{hi}CD8⁺ T cells. See for example, Verma et al. Nat. Immunol. 20, 1231-1243 (2019)). For example, CD8⁺ T cells can be contacted with one or more suboptimal antigens to produce PD1⁺CD38^{hi}CD8⁺ T cells. Exemplary antigens include, but are not limited to ovalbumin peptides, for example, Ova 257-264 (SIINFEKL) (SEQ ID NO: 1) or Ova-V, a low affinity variant of Ova 257-264, (SIIGFEKL) (SEQ ID NO: 2). As used herein, optimal priming refers to antigenic stimulation through T-cell receptors (TCRs) required by T cells to exert full expansion, effector functions and memory cell differentiation. In contrast, suboptimal priming refers to weaker antigenic stimulation through T cell receptors that is insufficient to maximize T cell expansion and/or functions and increases the number of dysfunctional T cells. For example, suboptimal priming results in a decrease of at least about 10%, 20%, 30%, 40%, 40%, 60%, 70%, 80%, 90% or 100% in one or more functions of a T cell as compared to priming/activation with an optimal antigen. For example, the T cell can be contacted with a suboptimal peptide or agent having decreased avidity for TCR, or decreased engagement of the TCR, as compared to a peptide or agent that optimizes T cell priming or activation, to increase the population of T cells that exhibit decreased function and/or expansion capabilities.

Blockade of PD-1 can comprise contacting CD8+ T cells with an agent that inhibits or disrupts the interaction of PD-1 and its ligand(s). In some methods, the agent inhibits the PD-1/PD-L1 and/or PD-1/PD-L2 pathway (i.e., the interaction of PD-1 with PD-L1 and/or the interaction of PD-1 with PD-L2). In some methods, a PD-1 inhibitor (e.g., an anti-PD-1 antibody) or a PD-L1 inhibitor (e.g., an anti-PD-L1 antibody) is used to produce a population of PD-1+CD38hiCD8+ T cells. It is understood that inhibition of the PD-1/PD-L1 and/or PD-1/PD-L2 pathway can also be referred to as PD-1 blockade.

Optionally, the CD8⁺ T cells can be contacted with IL-2 and/or an agent that inhibits PD-1 (e.g., an anti-PD-1 antibody) to induce production of PD1⁺CD38^{hi}CD8⁺ T cells.). In some cases, the T cells are contacted with IL-2 and an agent that inhibits PD-1. Optionally, induction of PD1⁺CD38^{hi}CD8⁺ T cells comprises contacting CD8+ T cells with a suboptimal antigen and an agent that inhibits PD-1 (e.g., an anti-PD-1 antibody or an anti PD-L1 antibody).

It is understood that methods for producing PD-1⁺CD38^{hi}CD8⁺ T cells can comprise stimulating, activating (i.e., priming) and/or differentiating CD8⁺ T cells in vivo, ex vivo or in vitro. Optionally, cells produced by these methods can be further purified, for example, by fluorescence activated cells sorting (FACS) for use in adoptive transfer of PD1⁺CD38^{hi}CD8⁺ T cells. PD1⁺CD38^{hi}CD8⁺ T cells can also be manipulated ex vivo to reduce the production of Gzm B in the cells before administration to the subjects. Any of the populations of PD-1⁺CD38^{hi}CD8⁺ T cells described herein or PD-1⁺CD38^{hi}CD8⁺ T cells produced by any of the methods described herein can be expanded prior to adoptive transfer.

### Methods of Treatment

Provided herein are methods for decreasing apoptosis of cells in a subject comprising administering to the subject an effective amount of an agent that decreases binding of a CD38-expressing cell to target cell, for example, a CD31-expressing cell in the subject. In some methods, apoptosis (i.e., cytotoxicity or cell killing) of CD31-expressing white blood cells is decreased in the subject. In some methods, apoptosis of CD31-expressing endothelial cells is decreased in the subject.

As used throughout, white blood cells are immune cells involved in protection against infectious disease and foreign antigens. White blood cells include, but are not limited to T lymphocytes, dendritic cells, natural killer cells and macrophages. As used herein, a T cell or T lymphocyte refers to a lymphoid cell that expresses a T cell receptor molecule. T cells include human alpha beta (ap) T cells and human gamma delta (γδ) T cells. T cells also include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. T cells can be CD4+, CD8+, or CD4+ and CD8+.

Provided herein are methods for decreasing apoptosis of cells in a subject comprising administering to the subject an effective amount of an agent that decreases binding of a CD38-expressing cell to a CD31-expressing white blood cell in the subject.

Also provided are methods for decreasing apoptosis of cells in a subject comprising administering to the subject an effective amount of an agent that decreases binding of a CD8⁺ T cell (e.g., a PD1⁺CD38^{hi}CD8⁺ T cell) to a CD31-expressing white blood cell in the subject. In some methods, the CD31-expressing white blood cell is a T cell, a dendritic cell, a natural killer T cell or a macrophage. In some methods, the binding between a CD8⁺ T cell (e.g., a PD1⁺CD38^{hi}CD8⁺ T cell) and one or more types of CD31-expressing white blood cells is decreased.

It is understood that the decrease in binding between the CD38-expressing cell and a CD31-expressing white blood cell is due, at least in part, to the binding of CD38, on the surface of the CD38-expressing cell, to CD31 on the surface of the CD31-expressing cell. As used throughout a decrease or reduction in binding does not have to be complete as the decrease can be a decrease of at least 10%, 20%, 30%, 40%, 50%, 60%, 70%, 80%, 90%, 100% or any percentage in between these percentages, for example, as compared to binding in the absence of an agent that inhibits the interaction between CD38 and CD31,or in the absence of reducing PD1⁺CD38^{hi} CD8⁺ T cells in the subject. Similarly, a decrease or inhibition of PD-1 (i.e., PD-1 blockade) does not have to be complete as the decrease can be a decrease of at least 10%, 20%, 30%, 40%, 50%, 60%, 70%, 80%, 90%, 100% or any percentage in between these percentages, for examples, as compared to the level of PD-1/PD-1 activity, in the absence of PD-1 blockade.

Agents that can be used to decrease binding of CD38 to CD31 include, but are not limited to a chemical, a small or large molecule (organic or inorganic), a protein, a peptide or an antibody. In some methods, the antibody specifically binds to CD38, for example, satuximab, daratumumab (Darzalex), and isatuximab (Sarclisa), to name a few. Additional anti-CD38 antibodies are described in U.S. Pat. Nos. 8,362,211, 8,088,896, 8,263,746, and 8,153,765. In some methods, the antibody specifically binds to CD31. In some methods, expression of CD38 in PD1⁺CD38^{hi}CD8⁺ T cells can be decreased ex vivo, for example, by contacting the cells with an inhibitory RNA or gene editing system, prior to administering the cells to the subject.

As used herein, the term antibody encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (K) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM. Several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. The term variable is used herein to describe certain portions of the antibody domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework region (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen but exhibit various effector functions, such as participation of the antibody in antibody-dependent cellular toxicity.

As used herein, an antigen binding fragment of an antibody refers to one or more portions of an antibody that contain the antibody's Complementarity Determining Regions (CDRs) and optionally the framework residues that include the antibody's variable region antigen recognition site, and exhibit an ability to specifically bind antigen. Such fragments include Fab′, F(ab′)₂, Fv, single chain (ScFv), and mutants thereof, naturally occurring variants, and fusion proteins including the antibody's variable region antigen recognition site and a heterologous protein (e.g., a toxin, an antigen recognition site for a different antigen, an enzyme, a receptor or receptor ligand, etc.).

In some methods, upon decreasing binding of CD38 to CD31-expressing cells, degranulation of (GzmB) in PD-1⁺CD38^{hi}CD8⁺ T cells is reduced in the subject. In some methods, upon decreasing binding of CD38 to CD31-expressing cells, transfer of GzmB from PD-1⁺CD38^{hi}CD8⁺ T cells into white blood cells in the subject is reduced. In some methods, PD-1⁺CD38^{hi}CD8⁺ T cells are manipulated ex vivo to reduce Gzm B prior to transplantation into a subject, for example, a subject having an autoimmune disorder.

Also provided are methods of decreasing apoptosis of white blood cells in a subject comprising decreasing the level of PD-1⁺CD38^{hi}CD8⁺ T cells in the subject. The level of PD-1⁺CD38^{hi}CD8⁺ T cells can be decreased, for example, by administering an agent that depletes PD-1⁺CD38^{hi}CD8⁺ T cells or by inhibiting induction of PD-1⁺CD38^{hi}CD8⁺ T cells in a subject. Agents that can be used to deplete PD-1⁺CD38^{hi}CD8⁺ T cells include, but are not limited to, a chemical, a small or large molecule (organic or inorganic), a protein, a peptide or an antibody. Immunomodulatory agents that bind to CD38 and/or PD-1 can be used to deplete PD-1⁺CD38^{hi}CD8⁺ T cells or to disrupt, inhibit, reduce or block PD-1 signaling. In some methods, a bispecific antibody that binds CD38 and PD-1 can be used to deplete CD38^{hi}PD-1⁺ T cells. The bispecific antibody is engineered to bind CD38 and CD8 on the same cell. Binding of the antibody to CD38 and PD-1 on the target cell can deplete the targeted cell.

PD-1⁺CD38⁺CD8⁺ T cells can also be depleted using a variety of ex vivo methods. For example, flow cytometry can be used. In such methods, immune cells are collected from the subject, for example in a biological specimen such as blood or from a tissue biopsy. For example, the immune cells are labeled with fluorescent antibodies against CD38, CD8, and/or PD-1, or combinations thereof. CD38^{hi}CD8⁺ T cells, CD38⁺PD-1 T cells, CD38-PD-1⁺CD8⁺ T cells or combinations thereof can be sorted out of the population of cells by a flow cytometer. The remaining population of cells can be administered back to the subject without the population of CD38⁺PD-1-CD8 T cells, thus decreasing the number of CD38⁺PD-1⁺CD8⁺ T cells in the subject.

In some methods, PD-1⁺CD38⁺CD8⁺ T cells are depleted using magnetic sorting. In these methods, immune cells are collected from the subject, for example in a biological specimen such as blood or from a tissue biopsy. The immune cells are labeled with magnetic nanoparticles-conjugated to antibodies against CD38, CD8, PD-1, or combinations thereof. CD3⁺CD8⁺ T cells, CD38⁺PD-1⁺ T cells, CD38⁺PD-1⁺CD8⁺ T cells or combinations thereof can be sorted out of the population of cells using a magnetic cell sorting device or column. The remaining population of cells can be administered back to the subject without the population of CD38⁺PD-1⁺CD8⁺ T cells, thus decreasing the number of CD38⁺PD-1⁺1⁺CD8⁺ T cells in the subject. In some methods, at least 10%, 20%, 30%, 40%, 50%, 60%, 70%, 80%, 90%, 100%, or any percentage in between these percentages, of cells are depleted in the sample from the subject.

In some methods, the subject has lung damage (for example, lung damage caused by acute or chronic lung inflammation), or ARDS. As used herein ARDS is a respiratory condition in which the lungs sustain a serious, widespread injury that diminishes their ability to provide the body's organs with enough oxygen. The condition causes fluid to accumulate in the lungs, which in turn reduces blood oxygen to dangerously low levels. Conditions that cause ARDS include, but are not limited to, pneumonia, sepsis, chest trauma, lung transplantation, cardiopulmonary bypass and viral infection of the lungs, including by SARS-CoV-2, the coronavirus that causes COVID-19 infection. Other ARDS-associated infections include, but are not limited to, influenza viruses, pneumonia, herpes simplex viruses, other coronaviruses, adenoviruses, measles viruses, mycobacterial tuberculosis, and cytomegalovirus. See, for example, Luyt et al. Presse Med 40(12): e561-e568 (2011); and Lee Int. J. Mol. Sci. 18(2): 388 (2011). Therefore, it is understood that any of the methods described herein comprising decreasing binding a PD-1⁺CD38^{hi}CD8⁺ T cell to a CD31-expressing cell (e.g., a white blood cell or an endothelial cell) can be used to treat lung damage or acute respiratory distress syndrome in a subject. Lung damage or ARDS can also be treated by reducing the number of PD-1⁺CD38^{hi}CD8⁺ T cells in the subject.

Also provided are methods for treating an autoimmune disease in a subject comprising administering to the subject a population of PD-1⁺CD38^{hi}CD8⁺ T cells. As used herein, an autoimmune disease is a disease where the immune system cannot differentiate between a subject's own cells and foreign cells, thus causing the immune system to mistakenly attack healthy cells in the body. Exemplary autoimmune diseases include, but are not limited to, inflammatory bowel disease, systemic lupus erythematosus, vasculitis, rheumatoid arthritis, Type 1 diabetes mellitus, myasthenia gravis, multiple sclerosis, psoriasis, Graves' disease, Hashimoto's thyroiditis, Sjögrens syndrome, and scleroderma.

Also provided are methods for treating an infection in a subject, comprising administering to the subject a population of PD-1⁺CD38^{hi}CD8⁺ T cells. In some methods, the infection is a non-ARDS-associated infection. The infection can be acute or chronic. An acute infection is typically an infection of short duration, while a chronic infection is a type of persistent infection that is eventually cleared. In the methods provided herein, an infection to be treated can be caused by a bacterium, virus, protozoan, helminth, fungal pathogens, parasitic pathogens or other microbial pathogens.

In any of the methods comprising administering PD-1⁺CD38^{hi}CD8⁺ T cells to a subject, the method can further comprise producing the population of PD-1⁺CD38^{hi} CD8⁺ T cells by contacting CD8⁺ T cells with a suboptimal antigen and/or a PD-1 inhibitor prior to administering the population of PD-1⁺CD38^{hi}CD8⁺ T cells to the subject. The suboptimal antigen that increases production of PD-1⁺CD38^{hi}CD8⁺ T cells can be selected by one of skill in the art based on the disease or disorder. In some methods, the suboptimal antigens are variants of antigens that are expressed by the pathological agents (e.g. SARS-COV-2, in the case of COVID-19).

In some methods, the PD-1⁺CD38^{hi}CD8⁺ T cells are expanded prior to administration to the subject. In any of the methods of treatment, the CD8⁺ T cells used to produce PD-1⁺CD38^{hi}CD8⁺ T cells can be autologous or autogeneic CD8⁺ T cells (i.e., from the same subject that receives the PD-1⁺CD38^{hi}CD8⁺ T cells); homologous or allogeneic (i.e., from a donor subject of the same species); or heterologous (i.e., from a different species). For allogeneic cells, CD8⁺ T cells can be isolated from a donor subject by obtaining a peripheral blood cell composition from the donor, depleting the peripheral blood cell composition of CD4⁺ T cells, natural killer cells etc. Optionally, the CD8⁺ T cell donor is HLA-matched, partially HLA-matched, or haploidentical to the recipient. In some methods the CD8⁺ T cells obtained from a subject can be cryopreserved prior to priming the cells with an antigen, for example, a suboptimal antigen, as described above. Optionally, the PD-1⁺CD38^{hi}CD8⁺ T cells produced using any of the in vitro or ex vivo methods described herein are cryopreserved prior to expansion and/or administration to the subject.

Any of the treatment methods described herein can further comprise administering an effective amount of a second therapeutic agent to the subject. The second therapeutic agent can be selected from the group consisting of a chemotherapeutic agent, an adjuvant, an immunomodulatory agent, an anti-infective (e.g., an antiviral, an antibacterial and the like), a vaccine, a potentiating agent, a pathogen antigen or a combination thereof. In some methods, the immunomodulator is an immunostimulant. As used herein an immunostimulant is an agent that stimulates or activates an immune response. Stimulating or activating an immune response includes inhibiting a suppressive immune response. Examples of immunostimulants include vaccines that can be used to stimulate an immune response.

In some methods, the immunomodulator is an immunosuppressant. As used herein, an immunosuppressant is an agent that suppresses or inhibits an immune response, for example, an immunosuppressant used to treat an autoimmune disorder.

Examples of immunosuppressants include, but are not limited to, calcineurin inhibitors (e.g., cyclosporin, tacrolimus), corticosteroids (e.g., methylprednisolone, dexamethasone, prednisolone) and cytotoxic inumunosuppressant (e.g., azathioprine, chlorambucil, cyclophosphamide, mercaptopurine, methotrexate).

In methods for treating ARDS-associated COVID or COVID, the second therapeutic agent can be selected from the group consisting of nirmatrelvir, ritonavir, remdesivir, molnupiravir, and an anti-SARS-CoV-2 monoclonal antibodies. As used herein, an immune response is the development of a beneficial humoral (antibody mediated) and/or a cellular (mediated by antigen-specific T cells or their secretion products) response directed against a peptide in a recipient patient. Such a response can be an active response induced by administration of an immunogen or a passive response induced by administration of antibody or primed T-cells. A cellular immune response is elicited by the presentation of polypeptide epitopes in association with Class I or Class II molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils, activation or recruitment of neutrophils or other components of innate immunity.

It is understood that combinations, for example, a composition comprising PD-1⁺CD38^{hi}CD8⁺ T cells and a non-cellular therapeutic agent described herein (for example, an immunomodulatory agent, an anti-infective, a vaccine etc.) can be administered either concomitantly (e.g., as an admixture), separately but simultaneously (e.g., via separate intravenous lines into the same subject), or sequentially (e.g., one of the compounds or agents is given first followed by the second). Any of the methods provided herein can further comprise surgery.

As used throughout, a subject can be a vertebrate, more specifically a mammal (e.g., a human, horse, cat, dog, cow, pig, sheep, goat, mouse, rabbit, rat, and guinea pig). The term does not denote a particular age or sex. Thus, adult, newborn and pediatric subjects, whether male or female, are intended to be covered. As used herein, patient or subject may be used interchangeably and can refer to a subject with or at risk of developing a disorder. The term patient or subject includes human and veterinary subjects. In any of the methods provided herein, the subject can be a subject diagnosed with a disease, for example, a respiratory disorder, an infection or an autoimmune disease.

As used herein the terms treatment, treat, or treating refers to a method of reducing one or more of the effects of the disorder or one or more symptoms of the disorder, for example, an autoimmune disorder, or a disease associated with respiratory distress in the subject. Thus in the disclosed methods, treatment can refer to a 10%, 20%, 30%, 40%, 50%, 60%, 70%, 80%, 90%, or 100% reduction in the severity of the disease or disorder. For example, a method for treating an autoimmune disorder is considered to be a treatment if there is a 10% reduction in one or more symptoms of the autoimmune disorder in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disorder or symptoms of the disorder.

As used herein, the term therapeutically effective amount or effective amount refers to an amount of a composition comprising PD-1⁺CD38^{hi}CD8⁺ T cells or cells differentiated therefrom, an immunomodulator, etc. described herein, that, when administered to a subject, is effective, alone or in combination with additional agents, to treat a disease or disorder either by one dose or over the course of multiple doses. A suitable dose can depend on a variety of factors including the particular cells or agent used and whether it is used concomitantly with other therapeutic agents. Other factors affecting the dose administered to the subject include, e.g., the type or severity of the disease. For example, a subject having multiple sclerosis may require administration of a different dosage of a composition comprising PD-1⁺CD38^{hi}CD8⁺ T cells or cells differentiated therefrom and/or an immunotherapeutic agent than a subject with lupus.

The effective amount of PD-1⁺CD38^{hi}CD8⁺ T cells or cells differentiated therefrom can be determined by one of ordinary skill in the art and includes exemplary amounts for a mammal of about 0.1×10⁵ to about 8×10⁹ cells/kg of body weight.

The effective amount of any compounds (for example, an immunomodulator or any other non-cellular therapeutic agent described herein) described herein or pharmaceutically acceptable salts or prodrugs thereof can be determined by one of ordinary skill in the art and includes exemplary dosage amounts for a mammal of from about 0.5 to about 200 mg/kg of body weight of active compound per day, which can be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. Other factors that influence dosage can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner. A therapeutically effective amount is also one in which any toxic or detrimental effects of the composition are outweighed by the therapeutically beneficial effects.

As used herein, administer or administration refers to the act of introducing, injecting or otherwise physically delivering a substance as it exists outside the body (e.g. PD-1⁺CD38^{hi}CD8⁺ T cells, cells differentiated therefrom or any non-cellular therapeutic agent described herein) into a subject, such as by mucosal, intradermal, intravenous, intratumoral, intramuscular, intrarectal, oral, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

Any of the therapeutic agents described herein (e.g., PD-1⁺CD38^{hi}CD8⁺ T cells, cells differentiated therefrom or any other non-cellular therapeutic agent described herein (for example, a vaccine, an adjuvant, an immunotherapeutic, etc.) are administered in a number of ways depending on whether local or systemic treatment is desired, and on the area to be treated. The compositions are administered via any of several routes of administration, including orally, parenterally, intramucosally, intravenously, intraperitoneally, intraventricularly, intramuscularly, subcutaneously, intracavity or transdermally. Administration can be achieved by, e.g., topical administration, local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; and European Patent Nos. EP488401 and EP 430539.

In some methods, a non-cellular therapeutic agent such as a small molecule, vaccine, an immunotherapeutic agent etc., can be delivered to the subject by way of an implantable device based on, e.g., diffusive, erodible, or convective systems, osmotic pumps, biodegradable implants, electrodiffusion systems, electroosmosis systems, vapor pressure pumps, electrolytic pumps, effervescent pumps, piezoelectric pumps, erosion-based systems, or electromechanical systems. Nanoparticle delivery is also contemplated herein. Effective doses for any of the administration methods described herein can be extrapolated from dose-response curves derived from in vitro or animal model test systems.

The cells and compounds described herein can be formulated as a pharmaceutical composition. In some embodiments, the pharmaceutical composition can further comprise a carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water.

The PD-1⁺CD38^{hi}CD8⁺ T cells or cells differentiated therefrom can be formulated as a pharmaceutical composition for parenteral administration. In some examples, the pharmaceutical composition further comprises a second therapeutic agent, as described herein. The T cells are typically administered in an aqueous solution, by parenteral injection. The formulation may also be in the form of a suspension or emulsion.

Depending on the intended mode of administration, a pharmaceutical composition comprising a non-cellular therapeutic agent described herein, can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

The examples below are intended to further illustrate certain aspects of the methods and compositions described herein and are not intended to limit the scope of the claims.

### EXAMPLES

### CD8⁺ T Cells Expressing PD1 and High Level of CD38 Generated by SOP are Unique CD8 T Cell Subtype

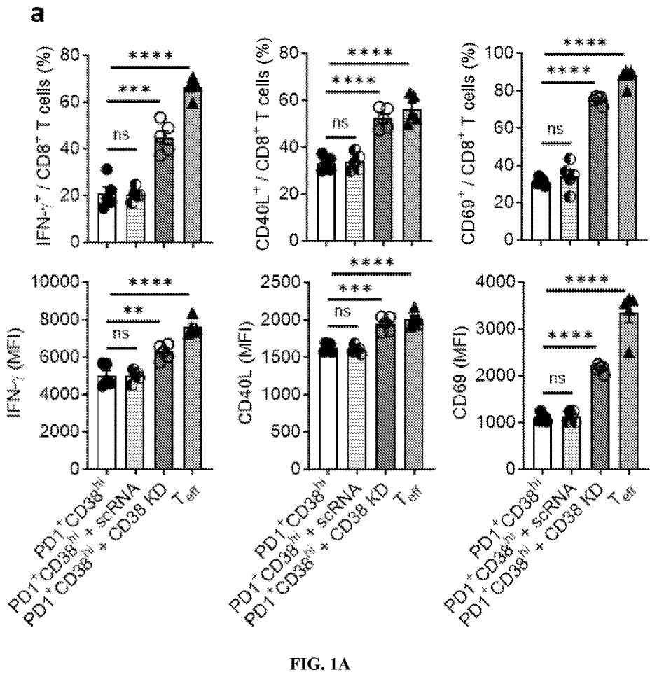

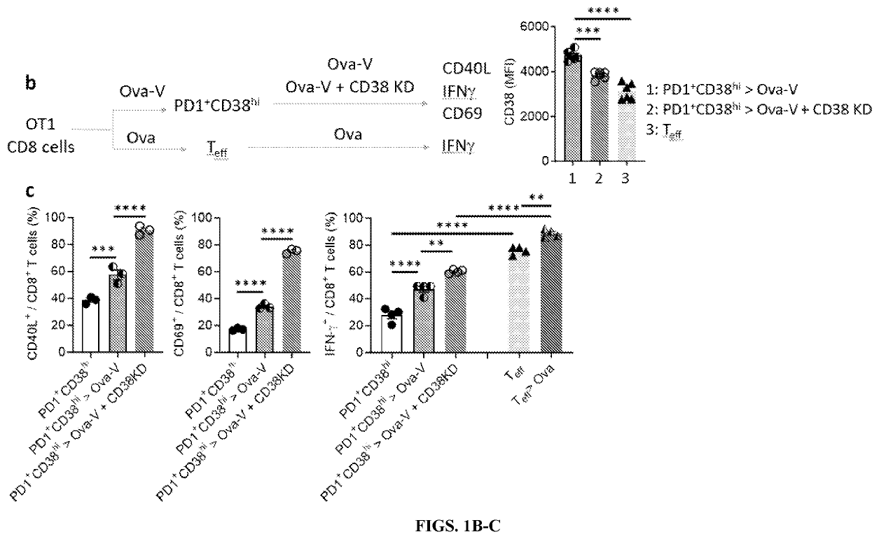

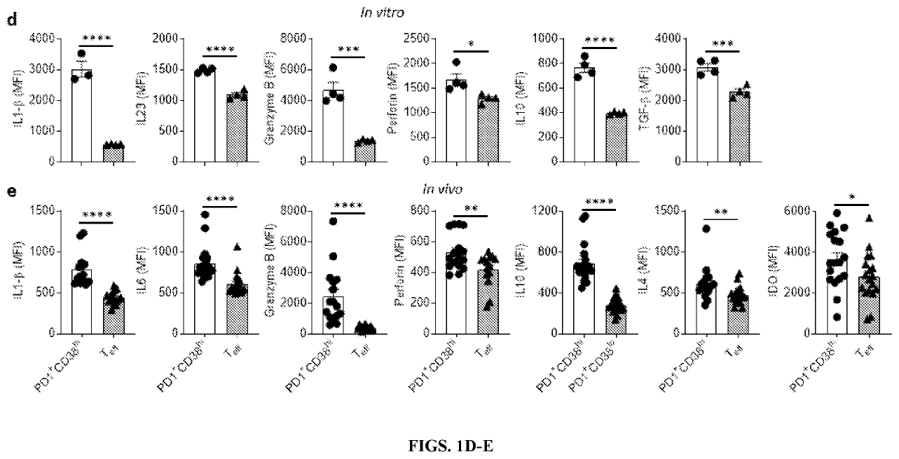

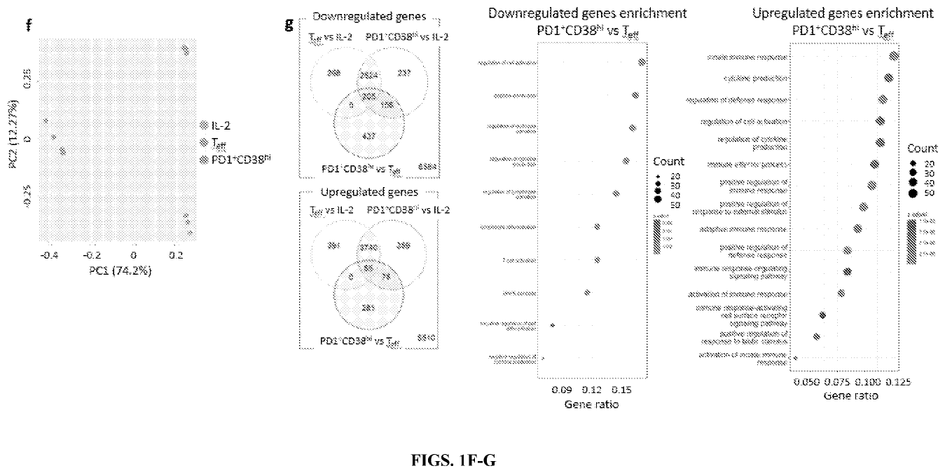

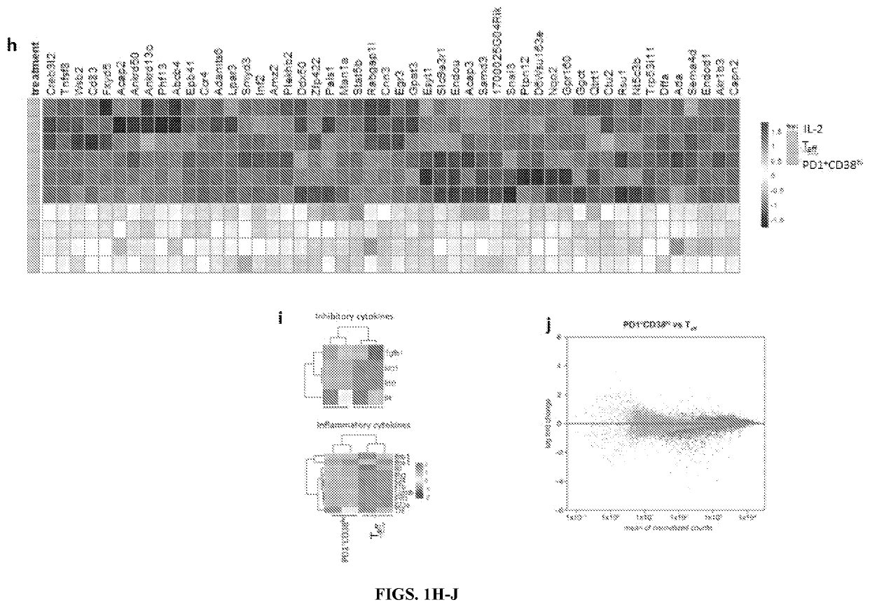

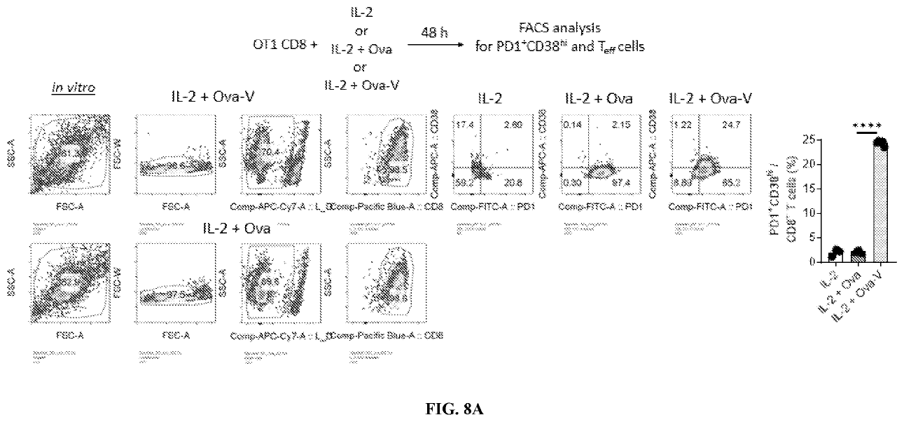

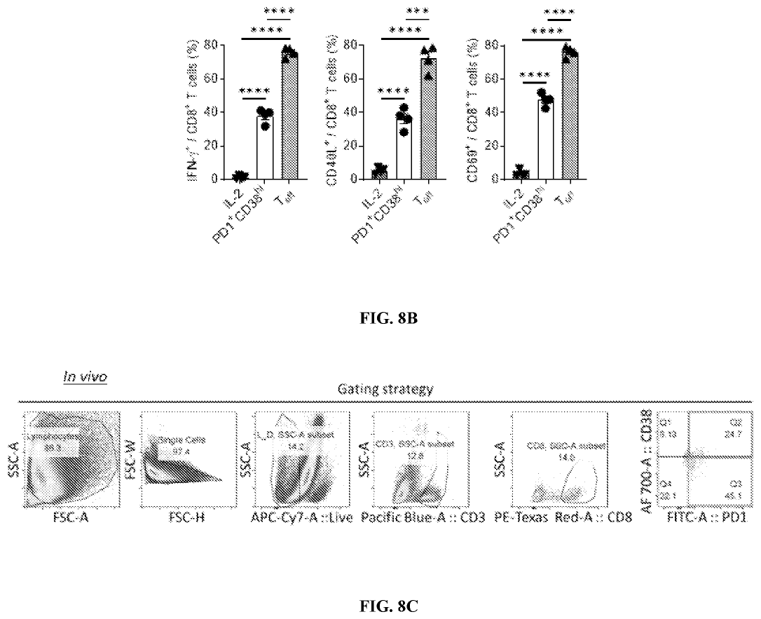

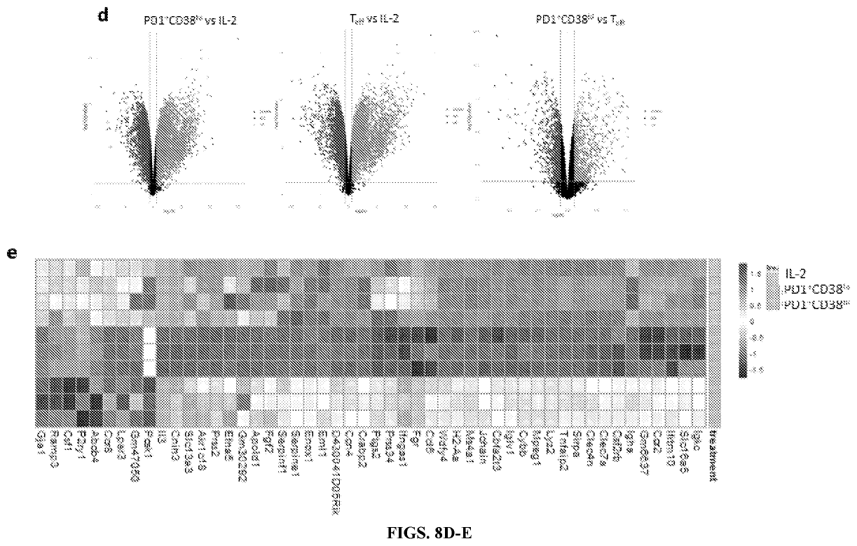

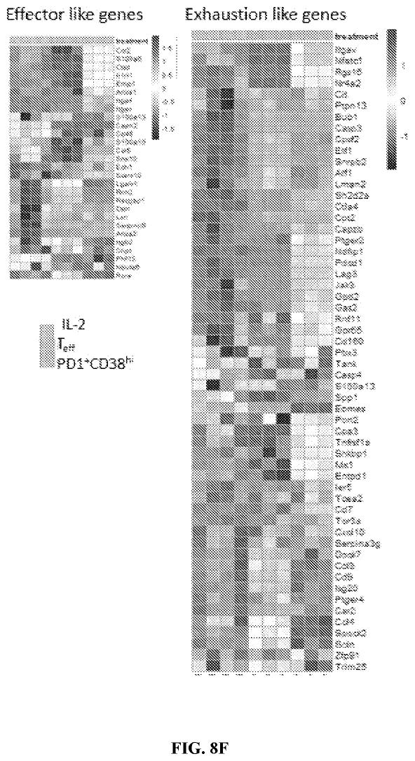

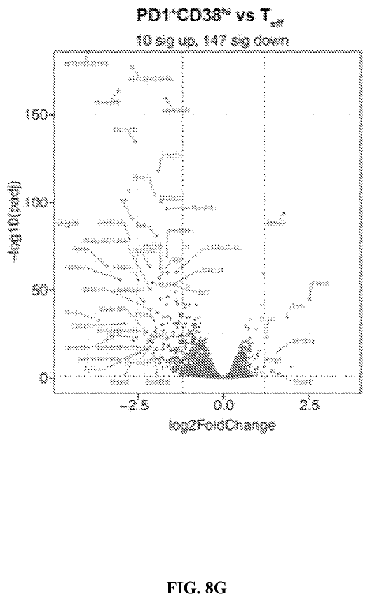

This laboratory previously reported that a novel subtype of CD8 T cells is induced under sub-optimal priming (SOP) T cell conditions in vitro and in the tumor microenvironment (TME) (Verma et al. Nat. Immunol. 20, 1231-1243 (2019)). These cells express PD1 and a high level of CD38 (PD1⁺CD38^{hi}) compared to T effector CD8 T cells (T_{eff}) and were found to play a role in resistance to anti-PD1 therapy. In the studies described herein, the cells were further characterized to understand their mechanism of action. To that end, the OT1 mouse system was used to generate T_{eff} CD8⁺ T cells by using WT OVA peptide or PD1⁺CD38 CD8⁺ T cells by SOP with low-affinity OVA-V peptide (Verma et al.). The priming was done for 48 hours under the same conditions (FIG. 8A). First, the activation status and function of these two-cell populations were compared. It was found that SOP-generated PD1⁺CD38^{hi}CD8⁺ T cells are dysfunctional as they express low level of IFN-gamma, CD40L, and CD69 (FIG. 1a & FIG. 8b). Since the low activation status of PD1⁺CD38 CD8⁺ T cells is CD38 dependent, here, CD38 was knocked down (KD) in these cells and their activation and function was compared directly to T_{eff}. It was found that KD of CD38 leads to recapitulation of their activation and effector function to a level comparable to T_{eff} cells as measured by the number of IFN-gamma⁺, CD40L⁺, and CD69⁺ cells (FIG. 1a). Interestingly, CD38 KD also enhanced the recall activity of PD1⁺CD38^{hi}CD8⁺ T cells after antigen rechallenge (FIG. 1b,c), though, it was still less than the recall activity of T_{eff} cells (FIG. 1c).

To further understand the function of PD1⁺CD38 CD8⁺ T cells, their cytokine profile was compared to T_{eff} cells. It was found that PD1⁺CD38^{hi} CD8 T cells produced significantly higher levels of both pro-inflammatory (IL-10, IL-23, IL-6) and anti-inflammatory (IL-10, TGF-β, IL-4, IDO) cytokines in vitro and in vivo when isolated from the TME of the TC-1 tumors (FIG. 1d,e). Interestingly despite their reduced activation and dysfunctional state, PD1⁺CD38^{hi} CD8 T cells produced significantly higher levels of Gzm B and perforin (Prf) in comparison to T_{eff} cells (FIG. 1d,e) (Gating strategy is outlined in FIG. 8a,c). These results suggest that SOP-driven PD1⁺CD38^{hi} cells are a unique subset of CD8 T cells that are distinct from T_{eff}cells. To confirm this, these cells were characterized at the transcriptome level by RNA-sequencing. Principal Component Analysis (PCA) of RNA-sequencing revealed that PD1⁺CD38^{hi} CD8 T cells have a distinct transcriptional profile when compared to both T_{eff} cells and the non-activated (IL-2) CD8 T cells (FIG. 1f). The difference between SOP-resulting PD1⁺CD38^{hi} CD8 T cells and optimally primed T_{eff} cells is mainly explained by PC2, indicating that PD1⁺CD38^{hi} CD8 T cells are a unique population that is not confined to a linear unprimed-to-properly primed axis, which may indicate their distinct status (FIG. 1f). Comparing PD1⁺CD38^{hi} to T_{eff} CD8 T cells, it was found that a significant change in 1,209 genes; 411 were upregulated and 798 were downregulated (p<0.001 & log₂ fold change >±1.2) (FIG. 1g Venn diagram, FIG. 8d). Since PC2 explains most of the differences between these two CD8 T cell populations, the top 50 differentially expressed genes (DEGs) associated with this axis were examined, as shown in FIG. 1h. A heatmap for the global top DEGs is shown in FIG. 8e. Next, signature genes that contribute to PD1⁺CD38^{hi} CD8 T cells' transcriptional distinctness were identified by comparing upregulated or downregulated genes for individual contrasts (that is naïve (IL-2), T_{eff} and PD1⁺CD38 CD8 T cells) (FIG. 1g Venn diagram). Utilizing this gene signature, a GO pathway enrichment analysis was performed to define the difference between PD1⁺CD38^{hi} CD8 and T_{eff} cells (FIG. 1g). Interestingly, PD1⁺CD38^{hi}CD8 T cells showed significant enrichment of upregulated genes in the immune effector process despite the enrichment of downregulated genes into T cell activation and differentiation pathways (FIG. 1g). Moreover, comparison of effector and exhaustion gene expression at the single gene level revealed many DEGs in PD1⁺CD38^{hi} compared to T_{eff} cells (FIG. 8f). However, overall enrichment at the gene set level was not observed (p<0.01). Furthermore, cytokine production and regulation pathways were also found to be enriched from both upregulated and downregulated genes, suggesting that regulation and the subsequent expression of cytokines are radically altered in PD1⁺CD38^{hi} CD8 T cells compared to T_{eff} (FIG. 1g). In line with these observations and in concordance with the protein data described above (FIG. 1d,e), it was found that expression of both the pro-inflammatory (Illb, 116, 1123) and anti-inflammatory (II10, tgfb, Ido, I110) cytokines were simultaneously upregulated in PD1⁺CD38^{hi} cells (FIG. 1i). Also in accordance with protein level, expression of Gzm B and Prf1 were higher than properly activated T_{eff} cells (FIG. 1i). Together, these data strongly suggest that PD1⁺CD38^{hi} CD8 T cells exist in a novel transcriptional state further indicating that these cells are a unique subset of CD8 T cells.

To understand this differential gene expression, ATAC-sequencing analysis was performed by comparing PD1⁺CD38^{hi} CD8⁺ T cells to the T_{eff} cells. The data shown in the log ratio (“minus”) vs average expression (MA) plot, the heatmap, and the volcano plot demonstrated that globally promoter-restricted peaks are less accessible in PD1⁺CD38^{hi} CD8 T cells compared to T_{eff} cells (FIG. 1j and FIG. 8g; FDR<0.05). Only 10 promoters were more accessible in PD1⁺CD38^{hi} vs Teffcells and 147 promoters were less accessible (FIG. 1j heatmap and FIG. 8g); FDR<0.05, |log 2FC|>1.2). Among the more accessible promoters in PD1⁺CD38^{hi} cells were Gzmk and T cell receptor gamma joining 1 and 4 (Trgj1, Trgj4) (FIG. 1j heatmap and FIG. 8g). The promoters that were less accessible in PD1⁺CD38^{hi} cells were enriched for pathways including cytokines and inflammatory response, cytokine-cytokine receptor interaction and JAK-STAT signaling (FIG. 1j heatmap). These data support a model of distinct regulation in PD1⁺CD38^{hi} cells with dampening of many T cell functions while a small subset are amplified. Together, the sequencing data demonstrate a closed chromatin structure in PD1⁺CD38^{hi} CD8⁺ T cells, suggesting that this may align with their reduced activated and differentiated state observed at the transcriptional level.

In sum, the high expression of functionally contrasting pro-inflammatory and anti-inflammatory cytokines at both RNA and protein level (total and active), differential epigenetic modifications, increased expression of cytotoxic proteins, expression of effector function-related genes despite a less activated and differentiated state, strongly support that PD1⁺CD38^{hi} CD8⁺ T cells exist in a state that is novel and distinct from previously documented cell states in the naive to T_{eff} cell paradigm. Furthermore, although PD1⁺CD38^{hi} CD8⁺ T cells express exhaustion-related genes they show an ability to recall after antigenic re-challenge unlike the terminally exhausted T cells, further confirming that these cells represent a unique cell phenotype.

Metabolic Characterization of PD1⁺CD38^{hi}CD8 T Cells

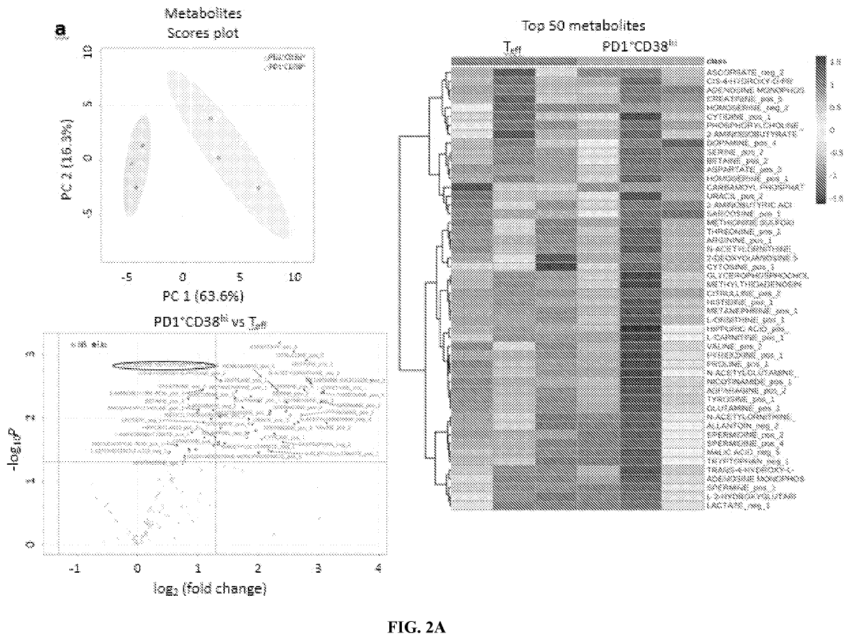

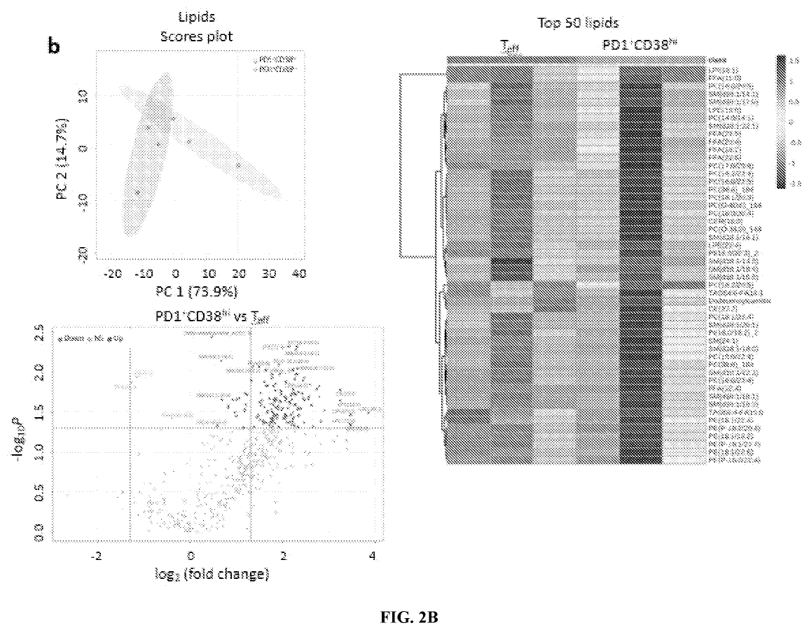

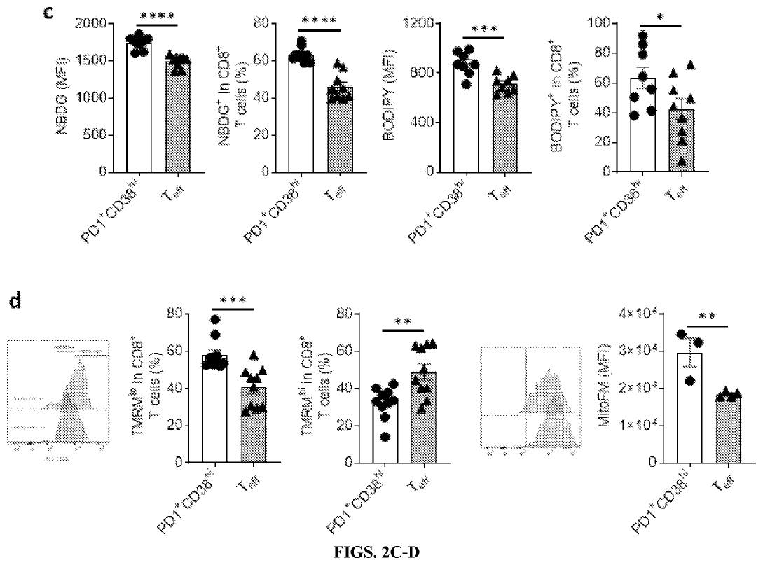

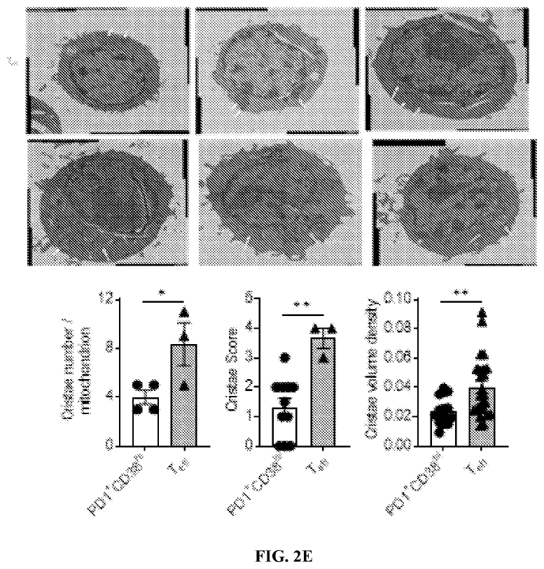

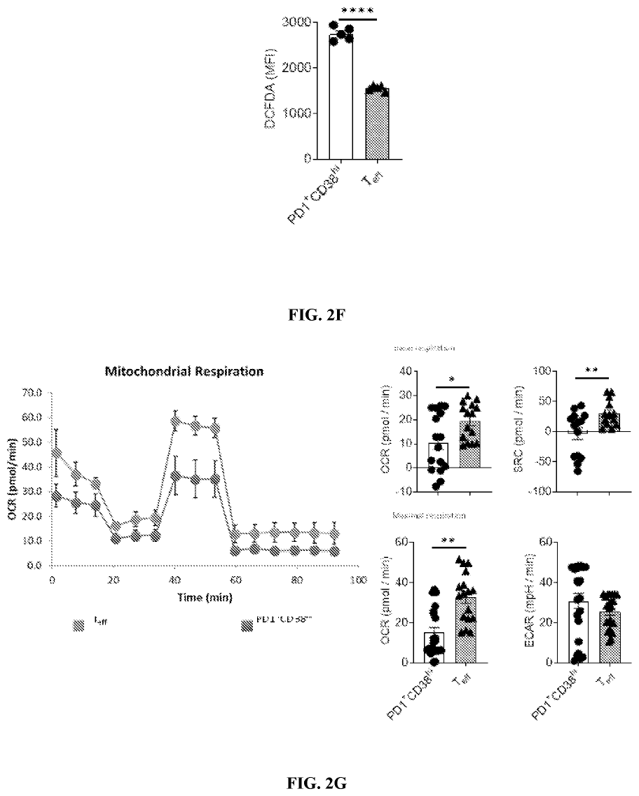

Since a unique CD8 T cell subtype was identified and, since it is well established that different T cell subtypes rely predominantly on specific metabolic pathways (glycolysis, lipid, oxidative phosphorylation, deep metabolomic (N=156) and lipidomic (N=615) analysis was performed to evaluate whether PD1⁺CD38^{hi}CD8 T cells are also metabolically distinct. Indeed, the PCA score, volcano plots and the heatmaps showed a distinct metabotype with a significant difference in 63 metabolites and 162 lipids in PD1⁺CD38^{hi} cells compared to CD8 T_{eff} cells (FIG. 2a,b). Interestingly, an unsupervised analysis showed that all of the top 50 metabolites, including AMP, lactate, aspartate, glutamine, alanine, were significantly increased in PD1⁺CD38^{hi} CD8 T cells (FIG. 2a,b). Similarly, except for 2 lipids (lyso-phosphatidylinositol (LPI) and free fatty acids (FFA)) out of top 50, all were significantly upregulated in these cells (FIG. 2a,b). Data above indicates that these cells are metabolically hyperactive. In accordance with metabolic hyperactivity, it was found that both glucose (NBDG) and fatty acid (FA) uptake (BODIPY staining) are significantly higher in PD1⁺CD38^{hi} CD8 T cells compared to CD8 T_{eff} cells (FIG. 2c). However, interestingly, despite the increase in mitochondrial mass correlating with the hyperactivity, these PD1⁺CD38^{hi} cells demonstrated dysfunctional mitochondria as seen by significant decrease in mitochondrial potential (FIG. 2d). Larger mitochondria with disorganized cristae were found as indicated by a lower cristae density, number and score (FIG. 2e), with a significant increase in the ROS production (FIG. 2f), further indicating that compared to T_{eff} cells mitochondria in PD1⁺CD38^{hi}CD8 T cells are structurally and functionally unfit. Furthermore, PD1⁺CD38^{hi} CD8⁺ T cells had a significantly lower oxygen consumption rate (OCR)/maximal and basal respiration and spare respiration capacity (SRC) with no significant change in extracellular acidification rates (ECAR) (FIG. 2g), indicating less energy production. Together, deep metabolomics and lipidomics, along with the findings from the metabolic assays, strongly suggest that PD1⁺CD38^{hi} CD8 T cells are metabolically distressed, utilizing all energy substrates.

PD1⁺CD38^{hi} CD8 T Cells are Fratricidal Cell that Kill in a Contact-Dependent Manner

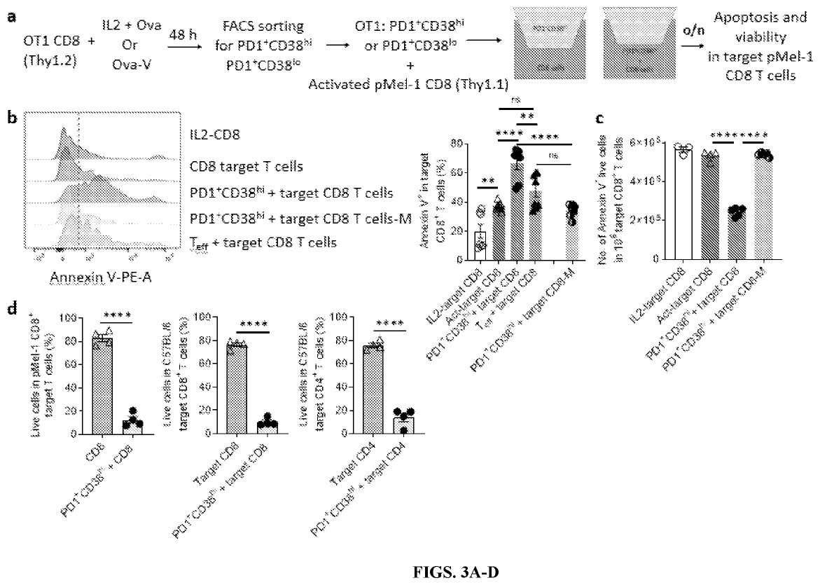

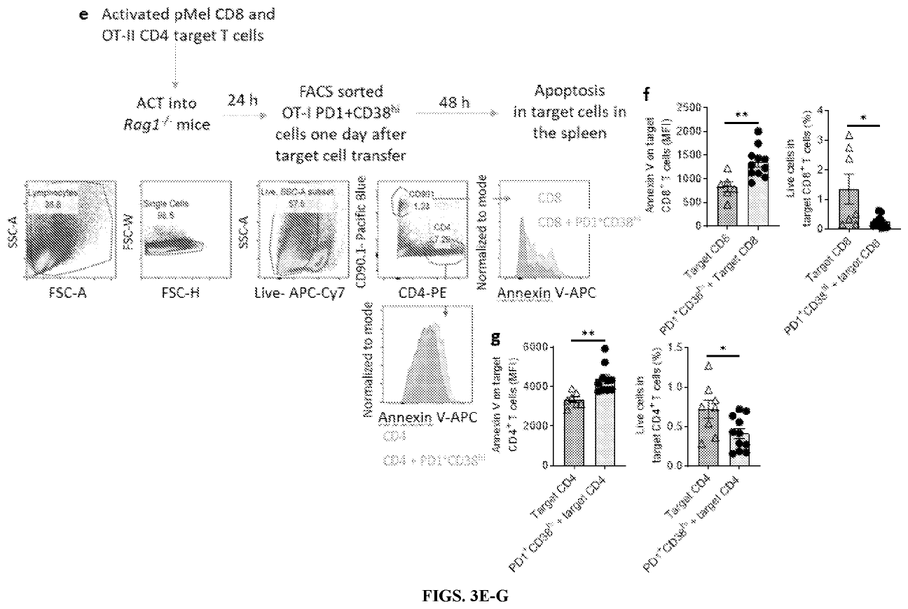

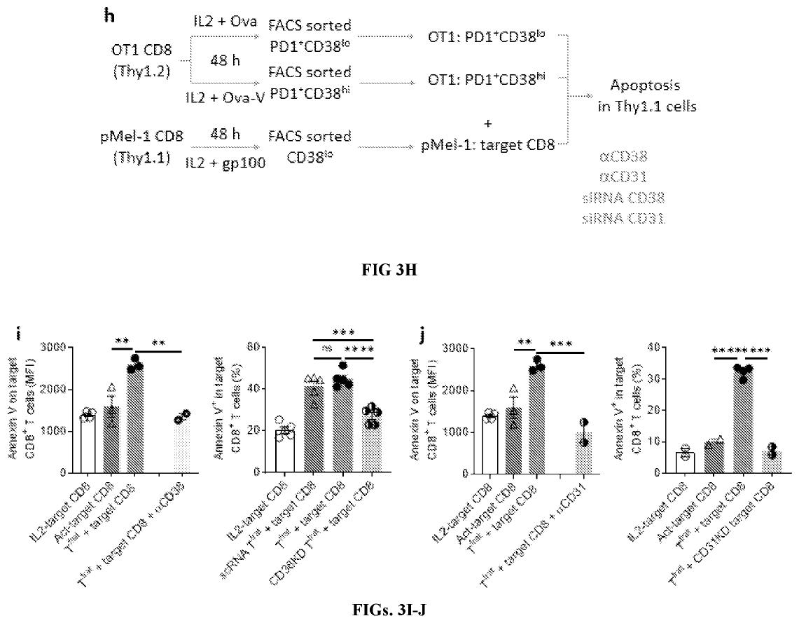

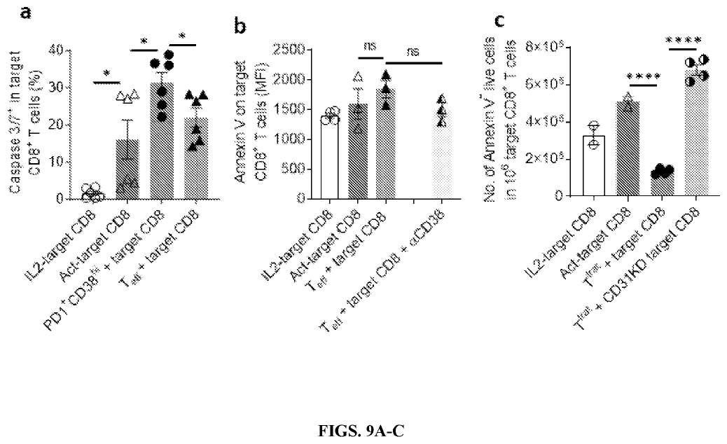

As outlined above, even though compared to T_{eff} cells, SOP-generated PD1⁺CD38^{hi} are dysfunctional, less activated, and metabolically distressed CD8 T cells, these cells possess high levels of Gzm B and Prf1. Therefore, it was hypothesized that these cells may have cytotoxic capability. To test this hypothesis, the ability of PD1⁺CD38^{hi} CD8 T cells derived from OT1 (Thy1.2) mice to induce apoptosis in target pMel-1 (Thy1.1) CD8 T cells in vitro (FIG. 3a) was checked. Interestingly, it was found that co-culturing PD1⁺CD38^{hi} CD8 T with target pMel-1 CD8 T cells strongly induces killing by apoptosis as indicated by annexin V binding and caspase3/7 staining, associated with significant decrease in the number of annexin V negative live pMel-1 target cells (FIG. 3b,c & FIG. 9a). On the other hand, as expected, CD8 T_{eff} cells do not induce such killing. The killing ability of PD1⁺CD38^{hi}CD8 T cells was also tested by a viability assay (live/dead cell staining) and it was found that 90% of pMel-1 target CD8 T cells were dead (FIG. 3d), further confirming PD1⁺CD38 CD8 T cells' killing capability. PD1⁺CD38^{hi} CD8 T cells were also tested for their ability to kill wild type (WT) CD8 and CD4 target T cells obtained from the spleens of C57BL/6 mice. Indeed, it was found that PD1⁺CD38^{hi} CD8 T cells kill both CD8 and CD4 T cells form WT mice, as shown by a significant decrease in the number of live target T cells in FIG. 3d. Accordingly, it was demonstrated that PD1⁺CD38^{hi} CD8 T cells are fratricidal T cells. To evaluate the mechanism of PD1⁺CD38^{hi} CD8 T cells-mediated killing, whether this killing is contact-dependent was tested. For this, PD1⁺CD38^{hi} CD8 T cells were mixed with target pMel-1 CD8 T cells or separated by a cell membrane using a trans-well chamber and stained for annexin V in the target cells and tested for viability (FIG. 3a). It was found that, indeed, this killing occurred in a contact-dependent manner, where PD1⁺CD38^{hi} cells induced apoptosis (annexin V) in target cells when mixed in suspension but not when the cells were separated with a cell membrane (FIG. 3b,c). Here, it is shown that these PD1⁺CD38^{hi} CD8 T cells exhibit their fratricidal activity in a contact-dependent manner.

To further confirm the fratricidal killing of PD1⁺CD38^{hi} CD8 T cells, whether these cells could kill autologous CD4 and CD8 T cells under in vivo murine setting was tested. For this, PD1⁺CD38^{hi} CD8 T cells generated from OT1 mice were transferred into Rag1−/− mice 24 hours after the mice were transfused with target cells (pMel-1-CD8 (Thy1.1) and OTII-CD4 T cells) (FIG. 3e). Transferring PD1⁺CD38^{hi}CD8 T cells into the Rag1−/− mice led to induction of apoptosis in both CD8 and CD4 target T cells, as shown by a significant decrease in the percentage of live target cells accompanied with increased MFI of annexin V on these cells (FIG. 3f,g). Accordingly, this further confirms the fratricidal ability of these cells to kill other T cells both in vitro and in vivo. The data above demonstrate that PD1⁺CD38^{hi} CD8 are fratricidal killer T cells (T^{frat}) inducing contact-dependent apoptosis in gp100-specific pMel-1 CD8 T cells and in WT autologous CD8 and CD4 T cells, demonstrating fratricidal activity in an antigen-independent manner.

### T^{frat} Kills Target Cells Through CD38:CD31 Interaction

As outlined above, T^{frat} cells induce their killing of target T cells in a contact-dependent manner (FIG. 3a-c). The mechanism by which T^{frat} cells induce contact-dependent apoptosis in target T cells was determined. Since these cells express high level of CD38, it was hypothesized that CD38-mediated signaling may be essential for this contact-dependent killing. To test the hypothesis, FACS-sorted T^{frat} cells were co-incubated with target pMel-1 CD8 T cells in the presence or absence of blocking CD38 antibodies, as outlined in FIG. 3h. As expected, T^{frat} cells but not T_{eff} cells led to induction of apoptosis in pMel-1 target CD8 T cells (FIG. 3i & FIG. 9b). However, it was found that blocking CD38 prevented the induction of apoptosis mediated by T^{frat} cells (FIG. 3i, left) while, as expected, blocking CD38 on T_{eff} cells did not affect target cell apoptosis (FIG. 9b). Similarly, genetic knockdown of CD38 also prevented T^{frat} cell-mediated apoptosis of the target cells (FIG. 3i, right). These results strongly suggest that T^{frat} cells induce their contact-dependent fratricidal effect through CD38.

It is known that CD31 is a natural ligand of CD38³, hence, it was hypothesized that T^{frat} cells may be inducing apoptosis through the interaction between CD38 and its non-substrate ligand CD31 on target T cells. To test this, killing assay, in the presence or absence of CD31 blocking antibody was performed, as described above. It was found that blocking CD31 prevents apoptosis in target cells when co-incubated with T^{frat} cells (FIG. 3j left). To further confirm the role of CD31 CD31 was genetically knocked down on target cells and it was found that the absence of CD31 also prevented the fratricidal activity of CD8 T^{frat} cells (apoptosis by annexin V binding) and resulted in an increase in the number of live target CD8 T cells (FIG. 3j right & FIG. 9c). These data prove that T^{frat} cells induce apoptosis in target cells through the direct CD38:CD31 interaction among the two cell populations.

T^{frat} Cells Kill by Transferring Gzm B into Target Cells

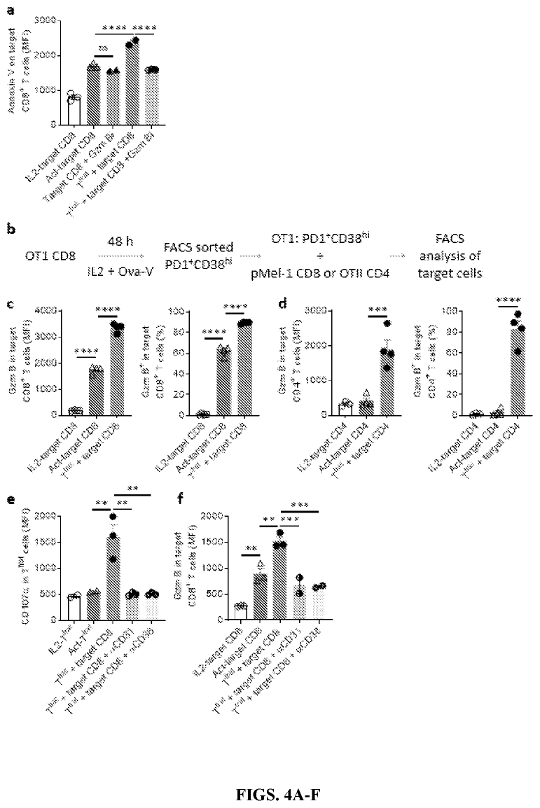

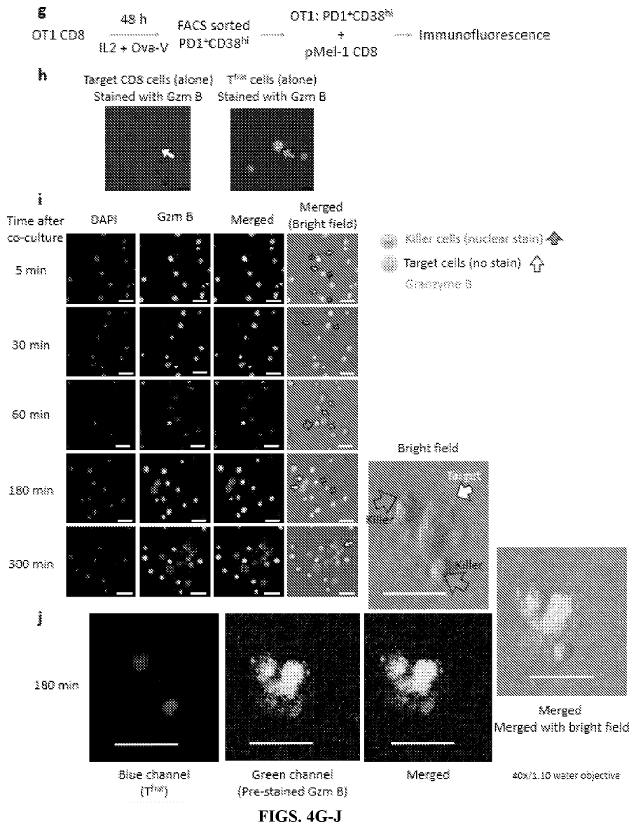

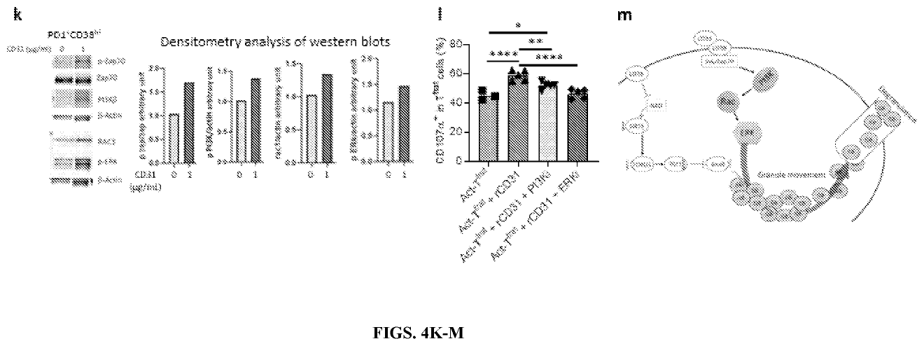

Next, the mechanism by which the T^{frat} cells induce apoptosis in the target T cells through the binding of CD38 to CD31 was investigated. Since T^{frat} cells produce high levels of Gzm B (FIG. 1d,e,i), whether the contact-dependent apoptosis mediated by these cells is Gzm B dependent was tested. It was found that when target pMel-1 CD8 T cells were incubated with T^{frat} cells in the presence of the Gzm B inhibitor, Z-AAD-CMK⁴, target cell apoptosis was fully abrogated, as shown by annexin V binding (FIG. 4a). This indicates a direct role of Gzm B in the induction of apoptosis in the target T cells. Next, whether T^{frat} cells induce killing through the direct transfer of Gzm B to target cells was checked. For this, T^{frat} cells were incubated with target pMel-1 CD8 T cells or target OTII CD4 T cells and the change in the level of Gzm B in the target cells pre- and post-incubation (FIG. 4b) was determined. A significant increase in the intracellular levels of Gzm B in the target cells (1.5 and 4-folds in target CD8 and CD4, respectively), as well as an increase in the frequency of Gzm B⁺ target cells from 60 to 90% in CD8, and from less than 1% to 80% in CD4 target cells (FIG. 4c,d), was found.

The data above could indicate that this increase in the level of Gzm B in target cells is due to triggering of Gzm B degranulation from the T^{frat} cells and transfer to target cells. To test this, T^{frat} cells were incubated with the pMel-1 CD8 target cells and the degranulation was estimated by the level of CD107a in T^{frat} cells. A significant increase in the expression levels of CD107a in T^{frat}, that is accompanied by enhanced Gzm B levels in target cells (FIG. 4e,f) was found. Importantly, degranulation in killer cells was CD38:CD31-mediated, since blocking the CD38:CD31 interaction by anti-CD31 or anti-CD38 resulted in a complete abrogation of degranulation in T^{frat} cells and complete loss of Gzm B increase in the target (CD8+) cells (FIG. 4e,f). Next, to prove that Gzm B is directly transferred from T^{frat} cells to target cells leading to their apoptosis, dynamic immunofluorescence analysis was performed by labeling Gzm B in DAPI-stained T^{frat} cells and incubated them as described above with target pMel-1 CD8 T cells (bright field, no staining) (FIG. 4g,h). It was found that target cells exhibited an enhanced uptake of labeled Gzm B from T^{frat} cells in a time-dependent manner, beginning from 60 minutes of co-culture of the two cell populations, as shown in FIG. 4i. FIG. 4j shows uptake of Gzm B by target cells after 3 hours of co-culture of T^{frat} and target cells at a higher magnification. These results conclusively show that the CD38:CD31 interaction results in degranulation of T^{frat} cells, leading to Gzm B-uptake and killing of target cells.

### CD38:CD31 Interaction Induces Gzm B Degranulation in T^{frrat} Cells Through Zap70-PI3K-RAC-ERK Pathway

As shown above, T^{frat} cells induced-target cell killing is mediated by CD38:CD31 interaction, which leads to degranulation and transfer of Gzm B from T^{a} to target cells (FIG. 4e-j). Next, molecular mechanisms by which ligation of CD38:CD31 leads to Gzm B degranulation were elucidated. Whether CD38 ligation with CD31 leads to an increase in the level of activated Zap70, resulting in an enhanced PI3K-RAC-ERK-mediated degranulation in T^{frat} cells, was investigated. For this, protein and phospho-protein levels of Zap70, PI3K, RAC, and ERK in T^{frat} cells were measured after incubation with recombinant CD31 (rCD31) protein using Western blotting. It was found that, upon incubation with rCD31 protein, there was an increase in the expression of phosphorylated Zap70, PI3K, RAC, and ERK in T^{frat} cells (FIG. 4k). To further confirm the involvement of this pathway in degranulation, the degranulation of Gzm B resulting from CD38:CD31 engagement, measured by CD107α level, in the presence or absence of PI3K and ERK inhibitors, was tested. Degranulation was inhibited in the presence of PI3K or ERK inhibitors (FIG. 4l). Thus, these results clearly demonstrate that CD38:CD31 interaction leads to induction of degranulation in killer cells through the activation of Zap70-PI3K-RAC-ERK signaling (FIG. 4m).

### Gzm B Production in T^{frat} Cells is Induced by CD38 Mediated-Depletion of NAD Through Modulation of SIRT1-FOXO1-TCF7 Pathway

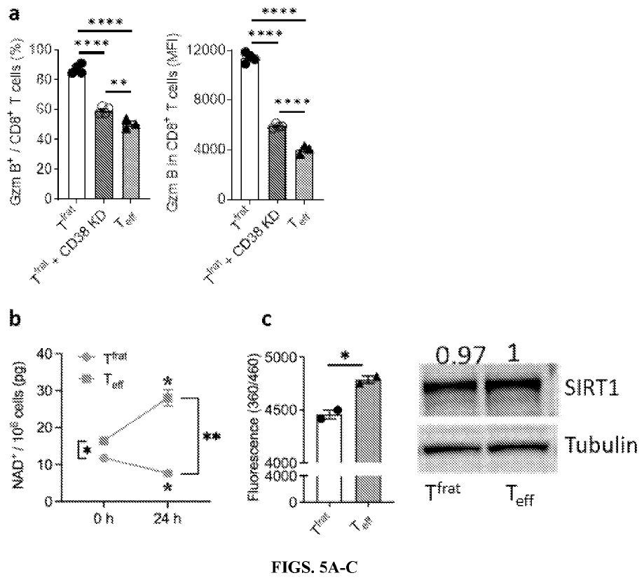

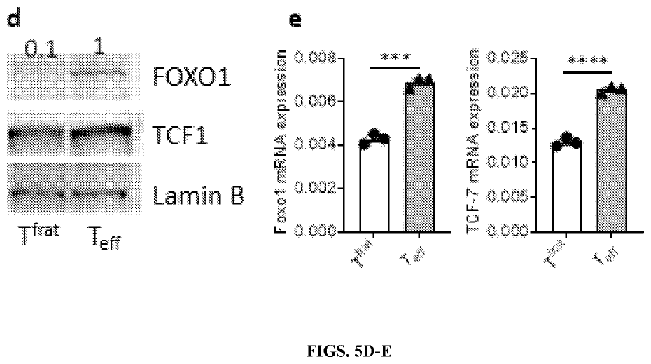

Since T^{frat} cells have a high level of Gzm B, compared to T_{eff} cells, and this is important for its fratricidal function, the mechanism by which these cells accumulate high levels of Gzm B and the potential role of CD38 in this process was investigated. For this, first, CD38 was knocked down in T^{frat} cells and this KD resulted in significant reduction in the level and frequency of cells expressing Gzm B, which is still higher relative to T_{eff} (FIG. 5a). Since the KD of CD38 was incomplete, this could explain the 50% reduction in Gzm B. Accordingly, this indicates that CD38 is essential for the accumulation of Gzm B in T^{frat} cells.

CD38 is an ectoenzyme that utilizes NAD as substrate ligand and NAD is required for the activity of the histone deacetylase, SIRT1, which leads to the acetylation and activation of FOXO1. Activated FOXO1, in turn, either directly or through TCF7, downregulates Gzm B production. Therefore, it was hypothesized that high expression of CD38 on T^{frat} cells leads to depletion of NAD, resulting in decrease in SIRT1 activity, FOXO1 and TCF7 levels, leading to enhanced expression of Gzm B. To test this hypothesis, the level of expression and activity of these molecules in T^{frat} was compared to T_{eff} cells. As shown in FIG. 5b, T^{frat} cells have reduced levels of NAD compared to T_{eff} cells at 0 h. Interestingly, NAD level is further reduced in T^{frat} cells, while it was increased in T_{eff} cells after 24 h of relevant antigen stimulation (FIG. 5b). CD38-mediated depletion of NAD in T^{frat} cells resulted in reduced SIRT1 activity with no change in protein level (FIG. 5c) and the protein and RNA level of FOXO1 and TCF7 (FIG. 5d,e) when compared to T_{eff} cells. To further confirm the link between the pathway and Gzm B accumulation, NAD was repleted or SIRT1 was activated using a specific activator, SRT1720 HCl, in T^{frat} and a decrease in the expression of Gzm B (FIG. 5 f,g) was found. Together, these results show that CD38 in T^{frat} cells mediates high expression of Gzm B by depleting NAD and in turn downregulating SIRT1-FOXO1-TCF7 axis (FIG. 4m). T^{frat} are induced in autoimmune diseases and exhibit an immune protective role

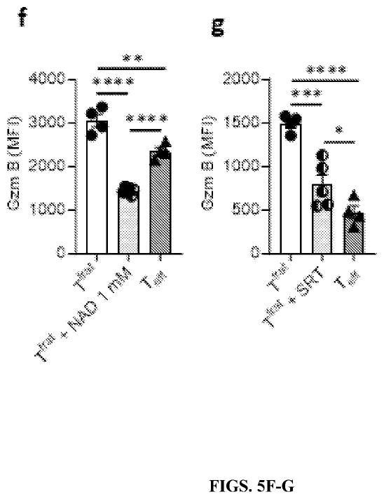

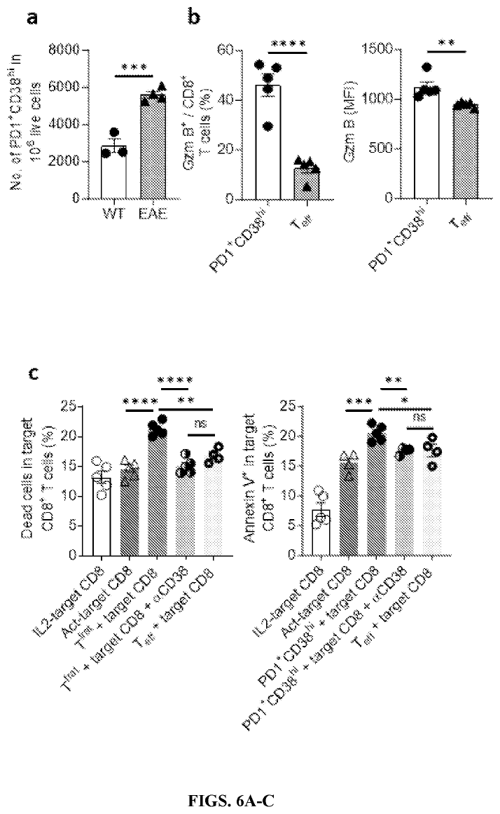

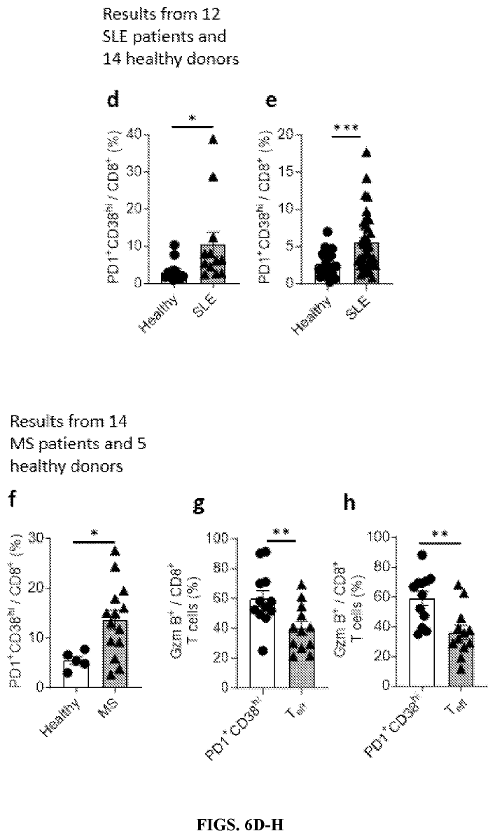

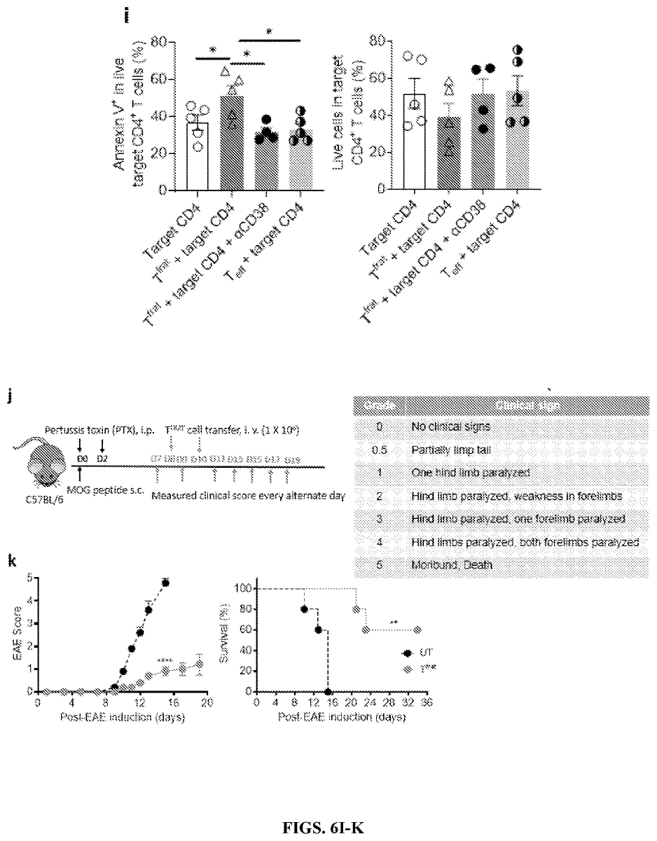

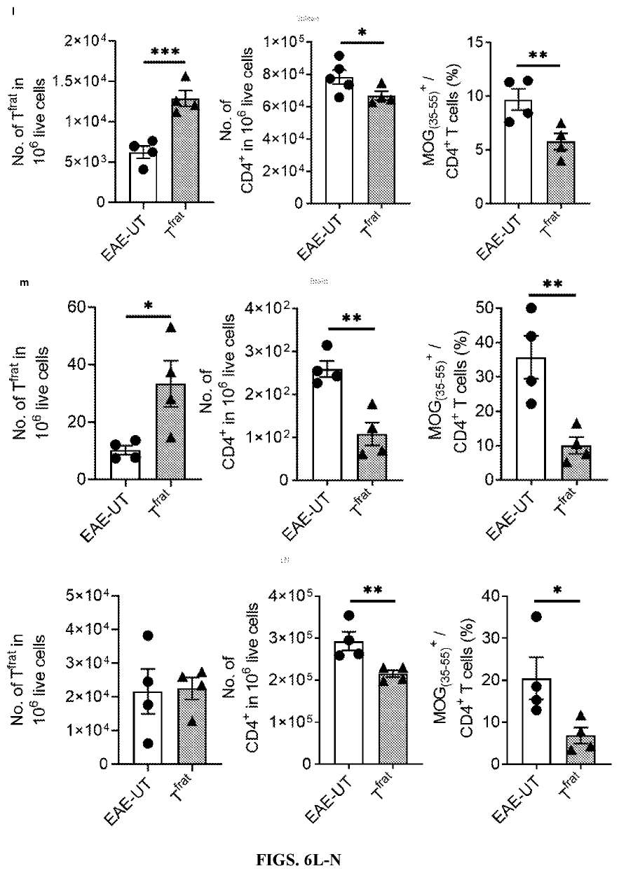

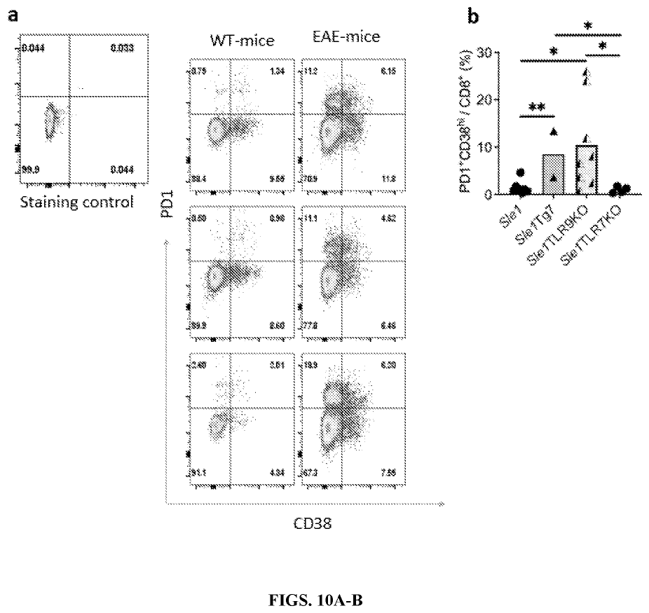

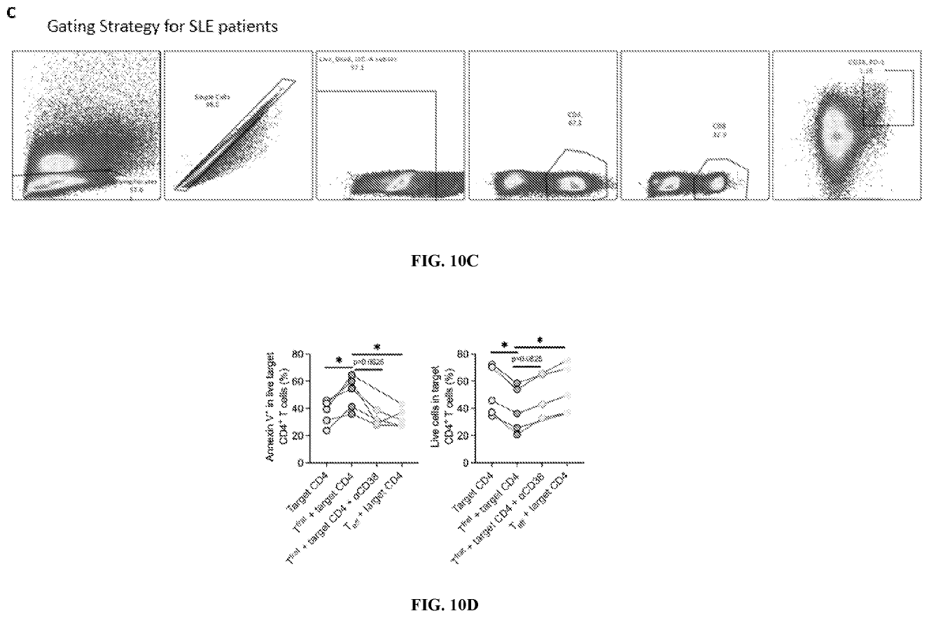

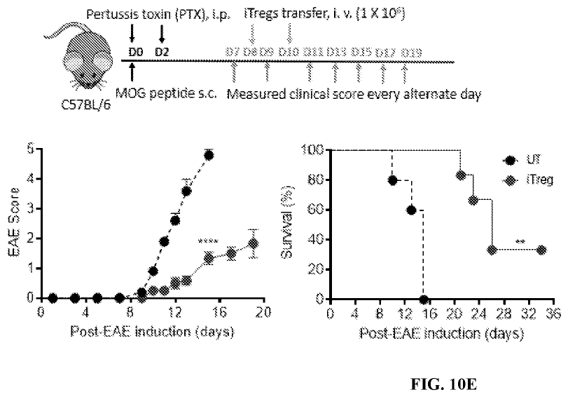

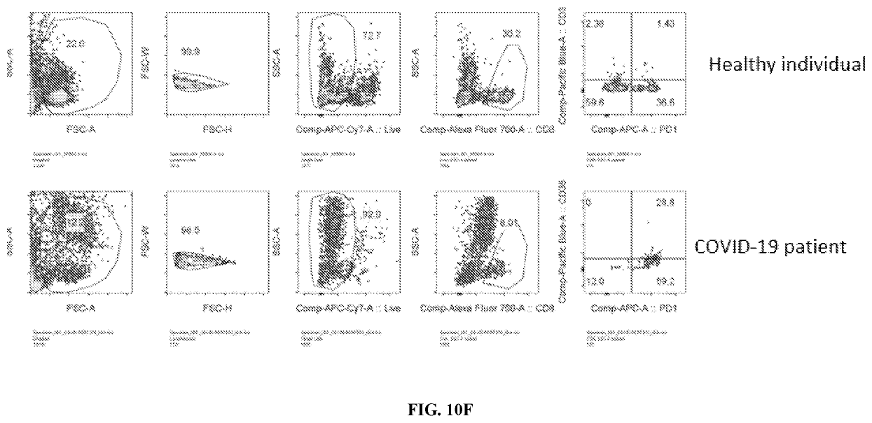

As T^{frat} are generated by SOP, and since autoimmune diseases (AIDs) are associated with induction of T cells against suboptimal self antigens, it was hypothesized that T^{frat} may be induced in AID. To that end, the level of PD1⁺CD38^{hi} CD8 T cells was tested in a mouse model of experimental autoimmune encephalomyelitis (EAE) (FIG. 10a). Interestingly, EAE mice demonstrated around a 3-fold increase in PD1⁺CD38^{hi} CD8 T cells compared to the WT mice (FIG. 6a). These cells express high levels of Gzm B compared to the T_{eff} cells obtained from the spleen of the EAE mice (FIG. 6b). To prove that these PD1⁺CD38^{hi} CD8 T cells are indeed T^{frat}, their ability to induce contact dependent fratricidal effect on pMel-1 target CD8 T cells was tested. PD1⁺CD38⁺CD8 T cells were isolated from the EAE mice at day 6 after disease induction and co-cultured with pMel-1 target CD8 T cells to test their fratricidal ability. Interestingly, these cells are indeed fratricidal, and they induce target CD8 T cells killing in a CD38-dependent manner since the blocking of CD38 reversed the killing (FIG. 6c). Similarly, a high level of PD1⁺CD38⁺CD8 T cells were also found in the SLE mice with severe disease (Sle1Tg7 and Sle1TLR9KO) compared to the mice with mild SLE disease (Sle1) (FIG. 10b).

To confirm the finding in human AID, the number of PD1⁺CD38^{hi} CD8 T cells in three cohorts of AID patients, 1 cohort of multiple sclerosis (MS) and 2 cohorts of systemic lupus erythematosus (SLE) (Gating strategy is shown in FIG. 10c) was analyzed. Patients with either of these AIDs have high numbers of PD1⁺CD38^{hi}CD8 T cells compared to the healthy individuals (FIG. 6d-f). As expected, these cells have a higher amount of Gzm B in the PD1⁺CD38^{hi} CD8 T cell subpopulation from patients with SLE (FIG. 6g) and MS (FIG. 6h) as compared to T_{eff} cells. To determine 10 if these cells behave as T^{frat}, the ability of PD1⁺CD38^{hi} CD8 T cells sorted from the SLE patients' PBMCs to induce contact dependent killing in autologous CD4 T cells, as targets, was tested. A significant increase in apoptosis of autologous CD4 T cells, when mixed with PD1⁺CD38^{hi} CD8 T cells, as measured by annexin V binding, with significant decrease in their viability (FIG. 6i & FIG. 10d), was found. It was also found that this killing was mediated by CD38, since its blockade by anti-CD38 significantly reduced the killing of autologous CD4 T cells (FIG. 6i & FIG. 10d). The data above demonstrates that these PD1⁺CD38^{hi} CD8 T cells induced in AID patients are indeed T^{frat} and could potentially explain lymphocytopenia in these patients.

Next, since the studies described herein showed these cells have an immune 20 suppressive role, it was hypothesized that T^{frat} induced under AID conditions may play an immune protective role in AID. To test this hypothesis, whether the infusion of T^{frat} cells in EAE mice inhibits autoimmune response and EAE outcome (FIG. 6j) was examined. Interestingly, the infusion of T^{frat}, prepared in vitro as described above, significantly reduced the clinical score and increased survival of EAE mice (FIG. 6k). This suppression of AID was comparable to the suppression demonstrated by Tregs (FIG. 10e). Next, to investigate if this suppression of AID was mediated by T^{frat} cell-mediated killing of activated T cells, the number of T^{frat}, total CD4, and MOG-specific CD4 T cells in various tissues from untreated EAE and EAE mice infused with T^{frat} cells were examined.

Studies showed the number of T^{a} were significantly increased in the spleen and brain compared to the untreated mice (FIG. 6l,m). Importantly, both the total number of CD4 T cells and antigen-specific (MOG) CD4 T cells are significantly decreased in spleen, and in target tissue brain and mesenteric LN in the EAE mice that received T^{frat} cells compared to untreated EAE mice (FIG. 6l-n). The data above clearly demonstrate that T^{frat} play a suppressive role for immune response and reverses AID.

Patients with Advanced COVID-19 have High Number of PD1⁺CD38^{hi} CD8 T Cells that Behave as T^{frat} and Kill Autologous T Cells

It has been reported that patients with advanced COVID-19 have high numbers of circulating PD1⁺CD38^{hi} CD8⁺ T cells, up to 30-60% of total CD8 T cells in the peripheral blood.

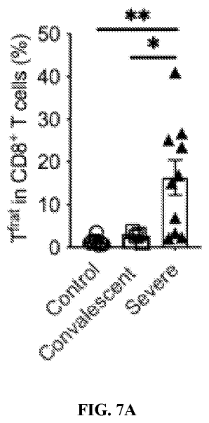

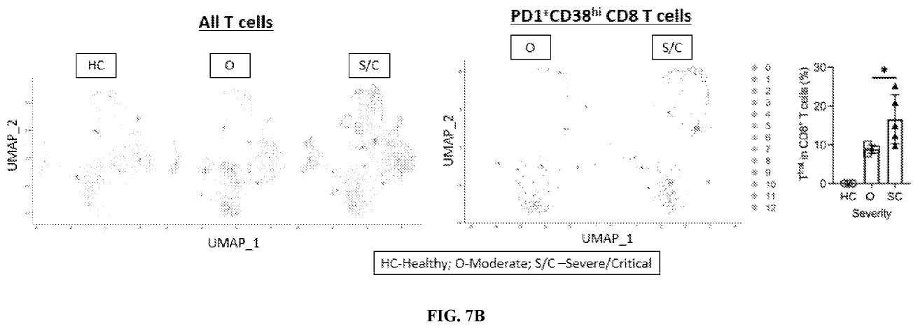

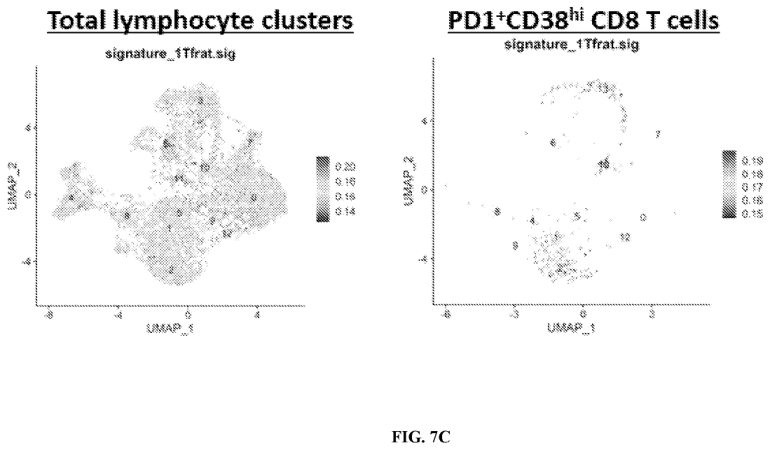

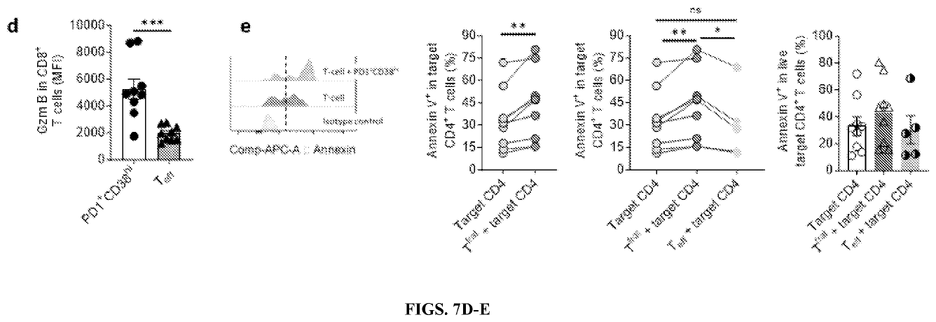

It was found that there are significantly higher numbers of PD1⁺CD38^{hi} CD8⁺ T cells in the blood of patients with advanced COVID-19 (n=11) compared to convalescent patients (n=5) or healthy individuals (n=11) (FIG. 7a and FIG. 10f). To further expand on this, publicly available single-cell RNA-sequencing data from bronchoalveolar lavage fluid (BALF) of COVID-19 patients was studied. Indeed, an increased number of PD1⁺CD38^{hi} CD8 T cells were found in the BALF of COVID-19 patients with severe/critical disease compared to moderate disease while they were absent in healthy individuals (FIG. 7b). To confirm that the PD1⁺CD38^{hi} CD8⁺ T cells in COVID-19 patients were phenotypically similar to T^{frat}, a subsetting and reclustering of BALF lymphocytes from the previously reported data was performed.

Unbiased clustering showed that CD38 was a signature marker for three CD8 clusters 1, 2, & 3. Further subsetting based on CD38 and PDCD1 expressing CD8 T cells defined that these cells indeed belong to same clusters 1, 2, or 3 (cluster 10 being only represented by one patient) (FIG. 7b). Coloring cells based on expression of signature gene sets for T^{frat} and T_{eff} as described above, revealed that cluster 2 was most transcriptionally similar to T^{frat} while cluster 3 was most similar to T_{eff} (FIG. 7c).

Accordingly, based on the findings that COVID-19 patients have a higher number of PD1⁺CD38⁺CD8 T cells with a gene signature similar to murine T^{frat} cells, it was hypothesized that PD1⁺CD38^{hi} CD8⁺ T cells in COVID-19 patients are T^{frat} which may also explain the severe lymphocytopenia observed in these patients. To demonstrate this, the levels of Gzm B were measured in the PD1⁺CD38^{hi} and T_{eff} CD8 subpopulations obtained from severe COVID-19 patients and it was found that PD1⁺CD38⁺CD8 T cells have high level of Gzm B compared to the T_{eff} cells (FIG. 7d). Whether these cells are capable of killing the patients' autologous lymphocytes was determined. Accordingly, the killing effects of PD1⁺CD38^{hi} CD8 T cells on patients' autologous CD4 T cells were tested. Indeed, it was found that incubating patients' PD1⁺CD38^{hi} CD8 T cells lead to induction of apoptosis in the autologous target CD4 T cells, as measured by annexin V staining in the host target lymphocytes (FIG. 7e). Accordingly, these significantly high numbers of PD1⁺CD38^{hi} CD8⁺ T cells in severe COVID-19 patients represent the T^{frat} cells, which may provide a plausible explanation for lymphocytopenia observed in these patients.

Bradykinin Induces PD-1⁺CD38^{hi} CD8⁺ Cells in a CREB-1 Dependent Manner

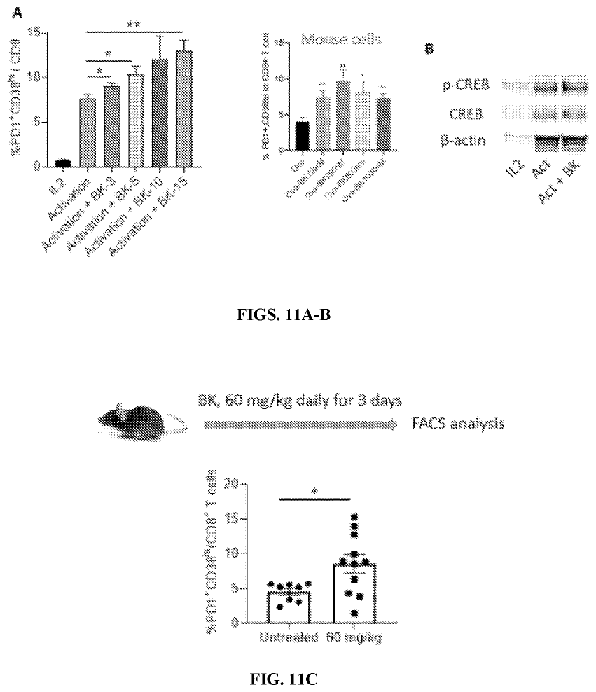

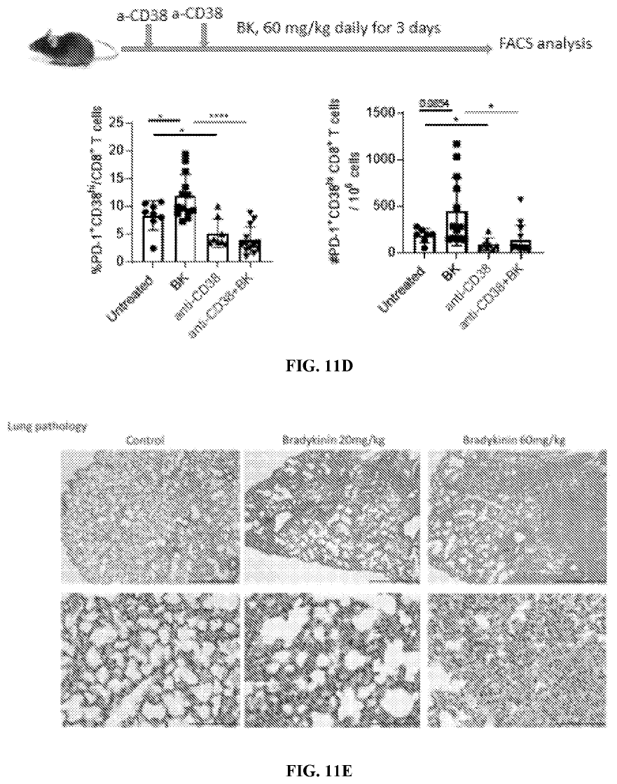

Advanced COVID-19 patients and acute respiratory distress syndromes (ARDS) are characterized by an unexplained severe lymphocytopenia and thrombosis. Due to lymphocytopenia, proportion of T cells, innate lymphoid cells and natural killer (NK) cells are significantly lower in severe disease compared to healthy donors. Moreover, COVID-19 patients are also characterized by a strong pro-inflammatory environment in multiple tissues. One of the pro-inflammatory factors found to be upregulated in these patients is bradykinin (BDK), also termed as BDK storm. BDK is a potent vasopressor that induces hypotension and vasodilation and is enhanced by the angiotensin produced by angiotensin-converting enzyme-2 (ACE2), the entry point for the SARS-CoV-2 virus. Interestingly, BDK activates cAMP response element-binding protein (CREB-1) with high potency, which in turn upregulates CD38 upon binding to CD38 promoter. Based on these observations, the BDK storm could lead to an increase in the number of PD1⁺CD38^{hi}CD8⁺ T cells and these cells could be one of the contributing factors for the observed pathophysiological conditions in COVID-19 patients and any ARDS related conditions. To test this hypothesis, either human or mouse CD8 T cells were treated with recombinant-BDK at various concentrations and interestingly, this treatment led to the generation of PD1⁺CD38^{hi}CD8 T cells (FIG. 11A). To further understand the BDK-mediated molecular mechanisms of induction of these cells, the level of phospho-CREB (p-CREB), a transcription factor that has binding elements in CD38 gene, was estimated in BDK-treated CD8 T cells. BDK signaling increased p-CREB expression (FIG. 11B). This induction of PD1⁺CD38^{hi}CD8⁺ T cells was inhibited when either BDK receptor or CREB was knocked-down by using specific siRNAs (FIG. 11C). Hence, these data show that BDK induces PD1⁺CD38^{hi}CD8⁺ T cells in a p-CREB dependent manner.

Since BDK mediates induction of PD1⁺CD38^{hi}CD8⁺ T cells under in vitro conditions, BDK may induce these cells in the lungs of mice (or under in vivo conditions), leading to ARDS (and its maintenance). Indeed, BDK administration in wild-type mice significantly increased the number of these killer cells in the lungs (FIG. 1D), which was reduced after CD38 depletion (FIG. 11E). Notably, this increase in the number of PD1⁺CD38^{hi}CD8⁺ T cells was accompanied with a severe lung pathology in BDK-treated mice. While the control tissue showed unremarkable lung parenchyma with intact alveolar spaces, lungs from BDK-treated mice demonstrated loss of open alveolar spaces with focal alveolar wall thickening compatible with alveolar damage (AD) (FIG. 11F). The septa were thickened with dense interstitial mixed inflammatory infiltrate compatible with interstitial pneumonia. Some alveolar walls showed focal pink material suggestive of hyaline deposits. Overall, the findings are compatible with features encountered in ARDS (proliferative phase).

PD1⁺CD38^{hi}CD8⁺ T Cells Kill Other Target Cells in a CD31 Dependent Manner

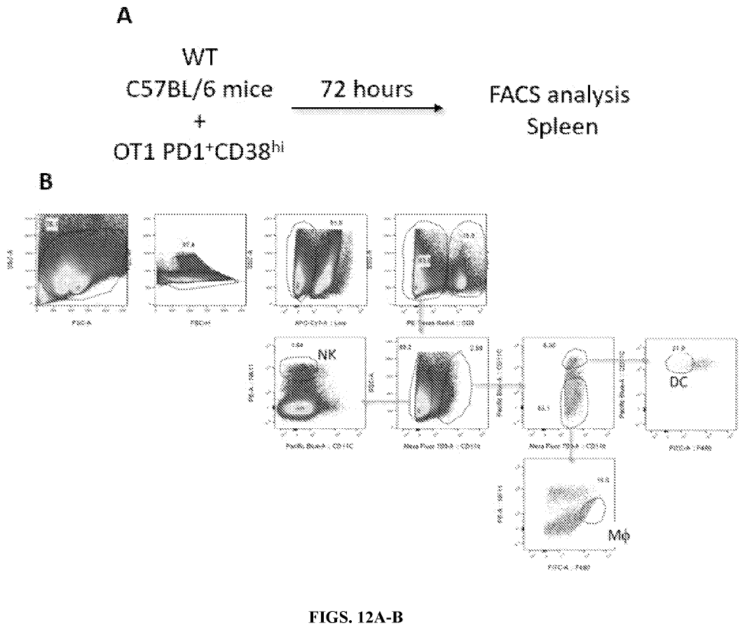

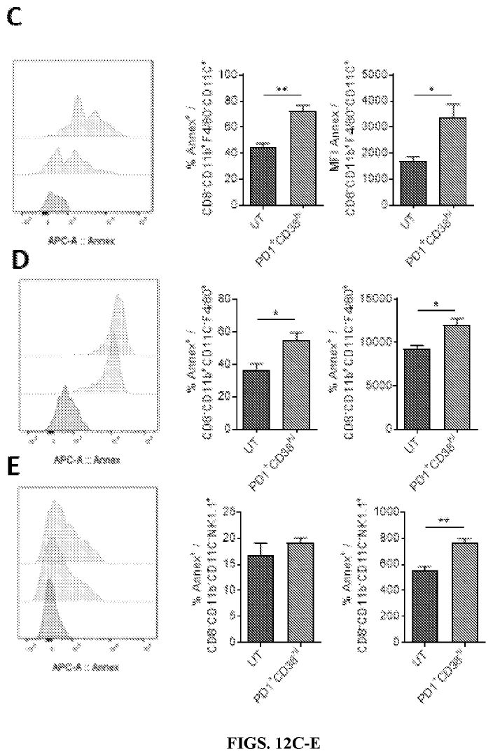

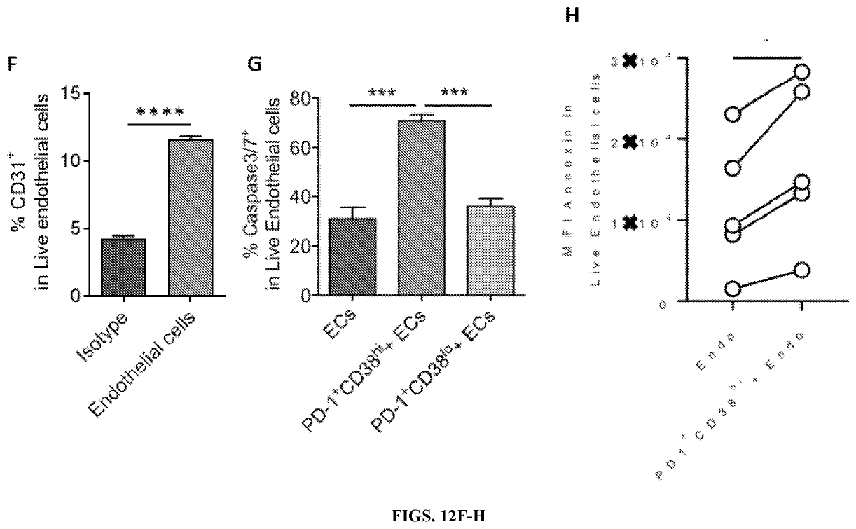

Since PD1⁺CD38^{hi}CD8⁺ T cells kill effector T cells through CD38:CD31 interaction, next whether these cells can kill other target cells that express CD31 such as myeloid cells (dendritic cells (DCs), macrophages), natural killer (NK) and endothelial cells (ECs) was assessed. For this, killing in DCs, macrophages and NK cells from spleen of WT mice that were treated with intravenous infusion of PD1⁺CD38^{hi} CD8⁺ cells was first estimated (FIG. 2A-B). The myeloid population isolated from mice that received PD1⁺CD38^{hi}CD8⁺ T cells showed higher apoptosis as compared to the control mice (FIG. 2C-E). Next killing of ECs by PD1⁺CD38^{hi} CD8⁺ T cells was estimated. C166 mice, mice from an EC line expressing high CD31 (FIG. 12F), showed increased apoptosis when incubated with the killer cells (FIG. 12G). Finally, testing was performed to determine whether PD1⁺CD38^{hi} CD8⁺ T cells from COVID-19 patients can also kill ECs. For this, human umbilical vein endothelial cells (HUVEC) were used, and, indeed, an enhanced killing of these cells resulted from PD1⁺CD38^{hi}CD8⁺ T cells from advanced COVID-19 patients (FIG. 12H). These results show that PD1⁺CD38^{hi}CD8⁺ T cells kill myeloid and endothelial cells, providing a plausible explanation for the observed thrombosis in COVID-19 patients and ARDS.

### Human Samples and Processing

PBMC samples from two cohorts of SLE patients and healthy donors (n=12) and 14 were collected at NCI: patients, n=12, healthy donors, n=14 and in Singapore: patients, n=35, healthy donors, n=25. PBMC samples from MS patients (n=14) and 5 healthy donors were obtained from the Center of Multiple Sclerosis and Autoimmune Neurology at the Mayo Clinic, Rochester, Minnesota. PBMC samples from COVID-19 patients (convalescent (n=5) and severe (n=10); (collected during acute phase of the disease) were collected at Providence Portland Medical Center (PPMC). Frozen PBMCs from COVID-19 patients were shipped to the Georgetown University on dry ice while experiments were performed at the respective sites for SLE and MS patients. PBMCs from healthy donors (n=8) were also purchased from Hemacare BioResearch Products & Services (Los Angeles, CA) (catalog no. M009C-2) for comparing with COVID-19 patient samples. PBMCs were thawed and stained with a LIVE/DEAD fixable near-IR dead cell stain kit (Invitrogen (Waltham, MA); catalog no. L10119) followed by staining with a cocktail of antibodies to the following surface markers: CD8, PD-1, and CD38¹ at a concentration of 1:200. Cells were fixed in the fixation buffer, permeabilized and then stained with appropriately labeled Gzm B antibody (1:100). Control stains were performed on each sample using isotype control antibodies to determine the marker positivity. Stained cells were acquired on an BD LSRFortessa (Franklin Lakes, NJ). Acquired samples were analyzed with the FlowJo software (FlowJo, LLC (Ashland, OR)). For the co-culture assays, PBMCs from the NCI SLE patient cohort and severe COVID-19 patients were stained with a cocktail of antibodies as mentioned above followed by PD1⁺CD38^{hi} CD8 T cells, T_{eff}, and CD4 T cells FACS sorting on FACS Aria™ III (BD Biosciences) into separate tubes. Sorted cell populations were used to perform co-culture experiments as detailed below.

The studies were performed in accordance with protocols, good clinical practice standards and the Declaration of Helsinki; protocols and all amendments were approved by the appropriate institutional review board or ethics body at each institution. All patients provided written informed consent.

### Mice

C57BL/6J (B6) and Rag1^{-/-} female mice, 4-6 weeks old, were purchased from The Jackson Laboratory (Bar Harbor, ME) or Charles River Laboratories (Charleston, SC). In-house-bred pMel-1 mice (B6.Cg-Thy1a/Cy Tg (TcraTcrb)8Rest/J) that carry a rearranged TCR transgene (Vpl13) specific for the mouse homolog (pmel-17) of human gp100²; OT I mice (C57BL/6-Tg(TcraTcrb)1100Mjb/Crl) that have transgenic TCR on CD8⁺ T cells specific for ovalbumin residues 257-264 in the context of H-2Kb; and OT-II mice (B6.Cg-Tg(TcraTcrb)425Cbn/J) having transgenic TCR on CD4⁺ T cells specific for ovalbumin residues 323-339 in the context of I-Ab were used as outlined in various experiments. In addition, the B6 mice carrying the lupus susceptibility region, Sle1 (B6.Sle1; defined by the microsatellite markers D1Mit17, D1Mit113, and D1Mit202), TLR9-deficient Sle1 mice (B6.S/e1TLR9KO), and conditional BAC Tg7 mice (Sle1Tg7) were bred at the Biological Resource Centre (Singapore). The derivation and the generation of these mice have been described previously³⁻⁶. All the mice were maintained under specific pathogen-free conditions. All procedures were carried out in accordance with approved Institutional Animal Care and Use Committee (IACUC) animal protocols at Georgetown University and the A*STAR IACUC approved protocol (#161176) which conforms to the NIH guidelines.

### Vaccines

Various cell types were activated with their respective cognate peptides. The gp10025-33 enneamer peptide (KVPRNQDWL (SEQ ID NO: 4)) purchased from AnaSpec was used for in vitro activation of magnetically enriched CD8⁺ T cells from the spleens of pMel-1 mice^{7,8} while CD8⁺ T cells from OT I mice were activated with either OVA₂₅₇₋₂₆₄ (SIINFEKL (SEQ ID NO: 1); catalog no. AS-60193-1; AnaSpec Inc.) or with a low-affinity variant of OVA₂₅₇-2₆₄, termed OVA-V (SIIGFEKL (SEQ ID NO: 1); catalog no. AS-64384; AnaSpec Inc.)². The target CD4⁺ T cells from the OTII mice were activated with H-2b-restricted Ova Class II epitope (OVA₃₂₃₋₃₃₉ (ISQAVHAAHAEINEAGR (SEQ ID NO: 3); catalog no. AS-27024; AnaSpec Inc. (Fremont, CA)). The purity of the enriched cells was >90%.

### Antibodies and Reagents

The fluorochrome labeled anti-mouse antibodies used for flow cytometry measurements were obtained from BD Biosciences, eBioscience (San Diego, CA), BioLegend (San Diego, CA), and ThermoFisher Scientific. Antibodies used for Western Blot were obtained from Cell Signaling Technology (Danvers, MA). Antibodies used for cell activation were: anti-mouse CD3 (5 μg ml⁻¹, clone 145-2C11, catalog no. 553057; BD Biosciences) and CD28 (2.5 μg ml⁻¹, clone 37.51, catalog no. 553294; BD Biosciences). CD38 and CD31 blocking antibodies used were CD38 (clone 90, Rat IgG2a; ThermoFisher Inc.) and anti-mouse CD31 (clone 390; REF #16-0311-85; eBiosciences) with isotype control antibodies (Rat IgG2a, K for anti-CD38 and anti-CD31. MOG₃₅₋₅₅/IA^{b} tetramers were obtained from MBL International Corp. (Woburn, MA).

CD8⁺ enrichment kits (Miltenyi Biotec (Germany)) were used according to the manufacturer's instructions. The Live/Dead Fixable Near-IR Dead Cell Stain Kit (catalog no. L34976) was obtained from ThermoFisher Scientific.

Recombinant CD31 (rCD31, 3628-PC-050, R&D) while Gzm B inhibitor (Z-AAD-CMK), PI3K inhibitor, ERK1/2 inhibitor (SCH772984), and SIRT1 activator (SRT1720HCl) were obtained from Selleckchem (Houston, TX). For measuring NAD levels, NAD/NADH Quantitation Kit (catalog no. MAK037, Sigma Aldrich (St. Louis, MO)) and for SIRT1 activity, SIRT1 activity assay kit (fluorometric, catalog no. ab156065, Abcam (Waltham, MA) were used. Primers and reagents for RT-PCR were purchased from Applied Biosystems (Foster City, CA). Myelin oligodendrocyte glycoprotein (MOG₃₅₋₅₅, catalog no. EK-2110), Complete Freund's Adjuvant (CFA, catalog no F588, Sigma), pertussis toxin (PTX, catalog no. BT-0105), and methylated bovine serum albumin (mBSA, catalog no.DS0162, EK-0133) were from Hooke Laboratories (Lawrence, MA).

For metabolomics and lipidomics, all liquid chromatography-mass spectrometry (LC-MS) grade solvents including acetonitrile and water were purchased from Fisher Optima grade, Fisher Scientific. High purity formic acid (99%) was purchased from Thermo-Scientific. Debrisoquine and 4-nitrobenzoic acid were purchased from Sigma-Aldrich. EquiSPLASH® LIPIDOMIX® quantitative mass spec internal standard and 15:0-18:1-d7-PA, C15 Ceramide-d7 (d18:1-d7/15:0) and 18:1 Chol (D7) ester were purchased from Avanti polar lipids. Internal standard for free fatty acid (FFA), dihydroceramides (DCER), hexosylceramides (HCER), lactosylceramides (LCER) were purchased from Sciex (Toronto, CA) as Lipidyzer platform kit.

siRNA for CD38 and CD31

For knockdown of CD38 and CD31, OTI (killer T_{frat} cells) and pMel-1 CD8⁺ T cells (target cells) were incubated with CD38 (catalog no. 4390771) and CD31 (catalog no. AM16708) siRNA respectively (10 μM; ThermoFisher Scientific) in 1% FBS medium overnight. SiRNA were prepared using Lipofectamine® RNAiMAX Reagent and OPTI-MEM® (both from ThermoFisher Scientific) per the manufacturer's recommendations. Next day, media containing Ova, Ova-V or gp100 (target cells, 1 μM) respectively were added to the wells for cell activation. Cells were collected after 48 h of incubation at 37° C.

### EAE Mouse Model

Experimental autoimmune encephalomyelitis (EAE) model in mice was established by earlier reported method^{9,10}. Briefly, MOG₃₅₋₅₅ (200 g/mouse) was mixed with equal amount of Complete Freund's Adjuvant (CFA). A total volume of 100 l/mouse was inoculated subcutaneously at day 0 in B6 mice. Pertussis toxin (PTX) 400 ng/200 l/mouse) was injected intraperitoneally on days 0 and 2. All animals were randomly assigned to each experimental group. Mice were observed daily for any signs of distress. For determining the number of PD1⁺CD38^{hi} CD8 T cells in EAE mice compared to the WT untreated mice, spleen were harvested from these mice at day 8 after induction of EAE. CD8 T cells were stained for PD1, CD38, and Gzm B and measured by flow cytometry. To check the fratricidal property of the PD1⁺CD38⁺CD8 T cells, FACS-sorted PD1⁺CD38^{hi} or T_{eff} CD8 T cells from the splenocytes of the EAE mice at day 6 after the disease induction were used to perform co-culture experiments as detailed below.

For ACT experiments, at days 8 and 10 after induction of EAE, mice received 1 million FACS-sorted PD1⁺CD38^{hi} CD8 T cells or FoxP3⁺CD4 regulatory (Treg) cells. For this, Tregs were generated from MACS-sorted CD4 T cells from splenocytes of B6 mice that were activated in T cell medium containing IL-2 (100 IU/mL), plate bound anti-CD3 (5 g/ml) and soluble anti-CD28 (2.5 g/ml) antibodies and TGF-β (2.5 ng/ml) for 72 h. Induced EAE symptoms were graded as reported earlier^{9,11}. For immune response experiments, mice were sacrificed two days after the second cell infusion and spleen, mesenteric lymph nodes and brain tissues were harvested, single cell suspensions were prepared and processed for estimation of different markers (CD8, CD4, PD1, CD38, and MOG₃₅₋₅₅) by flow cytometry.

SLE mouse model Single cell suspensions from the spleens of aged mice (6-9 months old) were prepared as described previously¹². Splenocytes were resuspended in PBS with 1% fetal calf serum (staining buffer) and then blocked for non-specific Fc binding using 20% 2.4G2 hybridoma supernatant. They were incubated on ice for 30 minutes with a master mix of antibodies to include CD45+ leukocytes, CD3⁺CD4+ and CD8⁺ T cells and CD38 and PD1 populations, together with a live/dead fixable dye (Biolegend). For intracellular TLR7 staining, the BD Cytofix/Cytoperm kit was used as per manufacturer instructions. Samples were analyzed using a BD FACSymphony A5.2 with Flowjo 10.9.0. Antibodies were purchased from BD Biosciences, ThermoFisher Scientific and Biolegend.

### Tumor Implantation and Mice Treatment

TC-1 tumor cell line generated from lung epithelial cells immortalized with 20 HPV16 E6 and E7 and h-ras oncogene were kindly provided by Dr. T-C Wu at Johns Hopkins University¹³. Cell line was routinely tested for absence of any contamination, including mycoplasma, by microscopic evaluation and PCR-based methods. Cells were cultured and tumors were implanted in B6 mice as we reported earlier^{7,8}. For assessing the expression of cytokines and cytolytic molecules, tumors were harvested from untreated mice when the tumor volume reaches 1.5 cm³ (day 18-20) after implantation. Samples were processed using a gentleMACS dissociator and the solid tumor homogenization protocol, as suggested by the manufacturer (Miltenyi Biotec). Single cell homogenate from the tumor sample was stained with appropriate antibodies as listed above and expression of various markers was checked by flow cytometry.

### Cell Activation, Treatments, and Recall Response

Primary murine CD8⁺ T cells as well as human CD8 T cells were isolated by fluorescence-activated cell sorting (FACS) and, in some cases, by negative selection using magnetic beads (Miltenyi Biotec), and cultured in RPMI 1640 medium supplemented with 10% FBS, 2 mM glutamine, 10 mM HEPES and 55 μM (3-mercaptoethanol. Purity of all the cell populations was greater than 95%.

PD1⁺CD38^{hi} and T_{eff} CD8 T cells were generated by our previously reported method². In brief, MACS purified (>90%) OT1-CD8 (CD45.1) T cells from mouse spleen were activated either with low-affinity OVA-V peptide or with high-affinity OVA peptide (1 M each) in T cell medium supplemented with 30 international units (IU) of IL-2 for 48 hours followed by FACS purification of PD1⁺CD38^{hi} and PD1⁺CD38^{lo} T cells respectively. Various cell surface and intracellular markers as well as level of pro- and anti-inflammatory cytokines, and cytolytic molecules were analyzed by flow cytometry as explained in the next section. In some experiments, CD38 was KD using siRNA for 24 h prior to various marker analysis. Furthermore, in a few experiments, either NAD (1 mM) or SIRT1 activator (1 or 2 μM) were added to the T_{frat} cells for 24 h and levels of Gzm B were measured in these cells and in T_{eff} cells by flow cytometry. To determine the role of CD38:CD31 interaction and signaling in Gzm B degranulation, T_{frat} cells were cultured with rCD31 (1 or 5 μg) with or without PI3K or ERK1/2 inhibitors (100 nM). After 24 h, degranulation of Gzm B in T^{frat} cells was measured by CD1071 staining and levels of various signaling molecules by flow cytometry.

For estimating antigen recall response, FACS-sorted T_{eff} and PD1⁺CD38^{hi} cells were re-challenged with respective peptides (OVA or OVA-V) for overnight and expression of IFN-7, CD40L, and CD69 was estimated by flow cytometry. In a few experiments, CD38 was KD in PD1⁺CD38^{hi} CD8 T cells for 24 h before the analysis of various markers.

For studies with BK, CD8 T cells from mouse spleen were activated with high-affinity OVA peptide (1 M each) and CD8 T cells from human were activated with anti-human CD3 (5 μg ml⁻¹, clone OKT3, catalog no. 317302; BioLegend) and CD28 (2.5 μg ml⁻¹, clone CD28.2, catalog no. 302902; BioLegend) in T cell medium supplemented (RPMI 1640 medium, 10% FBS, 2 mM glutamine, 10 mM HEPES and 55 μM P-mercaptoethanol) with 30 international units (IU) of IL-2. In some group various concentrations (murine: 50 nM-1000 nM; human: 3-15 μM) of recombinant-BK were added for 48 hours followed by FACS analysis of PD1⁺CD38^{hi} CD8 T cells.

### Flow Cytometry Analyses

For the flow cytometry analysis of lymphocytes of mouse and human origin, 1-2×10⁶ cells per sample were stained with the LIVE/DEAD fixable near-IR dead cell stain kit (Invitrogen; catalog no. L10119) followed by fixation and permeabilization. All surface and intracellular markers were stained at the same time in fix and per buffer as per manufacturer's recommendations. For IFN-7 and Gzm B staining, the BD Biosciences Cytofix/Cytoperm (catalog no. 51-2090KZ) and BD Biosciences Perm/Wash (catalog no. 51-2091KZ) buffer sets were used according to the manufacturer's instructions. In a few experiments, variously treated cells were processed for annexin V staining to check the extent of apoptosis. For this, collected cells were stained for annexin V in annexin binding buffer (ABB) (BD Biosciences) for 30 minutes at 4° C. After staining, samples were washed once in ABB and finally suspended, and FACS acquired in ABB as reported earlier¹⁴. For Caspase 3/7 staining, green flow cytometry assay kit (Thermo fisher catalog no. C10427) as per manufacturer's recommendations was used.

MitoFM, a green-fluorescent mitochondrial stain, which localizes to mitochondria and TMRM, the cell-permeant, cationic, red-orange fluorescent dye that is readily sequestered by active mitochondria and has been used for estimation of mitochondrial potential, was used as described previously¹⁵. Glucose uptake assay was performed using 2-NBDG⁸. All reagents were used as per the manufacturers' instructions. In a few experiments, the lipid profile of the PD1⁺CD38^{hi} and T_{eff} CD8 T cells was performed by BODIPY incorporation⁸. For this, PD1⁺CD38^{hi} and T_{eff} CD8 T cells were activated with Ova-V and Ova respectively for 48 h followed by surface staining as explained above. Cells were then washed and resuspended in 500 μl of BODIPY (493/503) prepared at a concentration of 0.5 μg ml⁻¹ in PBS. Cells were stained for 15 min at 20° C. followed by washing and final suspension in PBS. PD1⁺CD38^{hi} and T_{eff} CD8 T cells were stained similarly for DCFDA and analyzed by flow cytometry.

Data acquisition was performed on FACS Calibur or LSR Fortessa platform (BD Biosciences). Results were analyzed with the FlowJo software.

### Co-Culture (In Vitro Cell Killing) Experiments

PD1⁺CD38^{hi} and T_{eff} CD8 T cells were co-cultured overnight with FACS-sorted Thy1.1 target pMel-1 CD8⁺ T cells (killer to target cell ratio 1:1) that were activated with gp100 peptide (1 μM per 10⁶ cells per mL). To check the contact-dependent killing, a separation membrane (Corning Costar) was used and PD1⁺CD38⁺CD8 T cells were placed in the upper chamber on the membrane while the pMel-1 CD8⁺ target T cells were placed in the lower chamber. After overnight incubation, cells were harvested and apoptosis in target cells was estimated by annexin V staining. In a few experiments, CD38 was either blocked with anti-CD38 or KD in T^{frat} cells and CD31 was blocked with anti-CD31 or KD in the target pMel-1 CD8 T cells for 24 h followed by measurement of apoptosis by annexin V staining. In some experiments, Gzm Bi was added in the co-culture for 24 h followed by annexin V staining. In addition to the annexin V staining, Gzm B levels in the pMel-1 target CD8 or CD4 T cells and CD1107a levels in the T_{frat} cells after their co-culture in the presence or absence of anti-CD38/CD31 were measured by flow cytometry. In vitro cell killing experiments were also conducted after co-culture of PD1⁺CD38^{hi} CD8 T cells with target CD8 T cells obtained from the wild-type B6 mice. Viability of target cells was assessed by FACS analysis using fixable Live/Dead stain.

FACS-sorted PD1⁺CD38^{hi} or T_{eff} CD8 T cells from the EAE mice were co-cultured with target pMel-1 CD8 T cells for overnight. In some experiments, anti-CD38 was added to the T^{frat} cells during their-co-culture with the target CD8 T cells. Similarly, FACS-sorted PD1⁺CD38^{hi} or T_{eff} CD8 T cells isolated from the PBMCs of the SLE and COVID-19 patients were co-cultured with autologous target CD4 T cells with and without anti-CD38. Apoptosis induction by annexin V staining and cell viability by live/dead staining was measured by flow cytometry in the target T cells.

To check the killing of endothelial cells by PD1⁺CD38^{hi} CD8 T cells, C166, a mouse endothelial cell line expressing high CD31 was used. First, the expression of CD31 on these cells was determined by flow cytometry using anti-mouse CD31 (clone JC/70A; catalog: MA5-13188; eBiosciences) with isotype control antibodies (Mouse IgG1a, K, MA1-10406; eBiosciences). Next, PD1⁺CD38^{hi} CD8 T cells were co-cultured with these cells and apoptosis was determined by caspase3/7 staining using green flow cytometry assay kit (Thermo fisher catalog no. C10427) as per manufacturer's recommendations in target endothelial cells by flow cytometry. In addition, FACS-sorted PD1⁺CD38^{hi} CD8 T cells isolated from the PBMCs of the COVID-19 patients were co-cultured with target human umbilical vein endothelial cells (HUVEC) for 24 h followed by measurement of apoptosis by annexin V staining in target endothelial cells by flow cytometry.

### Adoptive Cell Transfer and In Vivo Killing Experiment

pMel-1 CD8 T cells activated with gp-100 (1 μM) or Ova-activated (1 μM) OTII-CD4 target T cells were intravenously transfused into Rag1−/− mice. One day later, PD1⁺CD38^{hi} CD8 T cells from OT1 mice (T_{frat}) generated as explained above, were transferred intravenously into these mice. Forty-eight hours later spleen were harvested and the levels of apoptosis in the target cells was estimated by Annexin V staining of CD90.1 CD8 or CD4 target T cells.

In another set of experiments, PD1⁺CD38^{hi} CD8 T cells generated as above, were transferred intravenously into C57BL/6J (B6) mice. Forty-eight hours later spleen were harvested and the level of apoptosis in the target cells that express CD31 (dendritic cells (DCs), macrophages CD11b+, F4/80+), natural killer (NK1.1) and endothelial cells (ECs) was estimated by Annexin V staining.

BK-Mediated Induction of PD1⁺CD38^{hi} CD8 T Cells In Vivo

To investigate if BK mediates induction of PD1⁺CD38^{hi} CD8 T cells under in vivo conditions, C57BL/6J (B6) mice were injected intravenously with BK from Siga Aldrich (60 mg/kg, daily) for 3 days and after 48 hours mice spleen were harvested, CD8 T cells were stained for PD1, CD38, and measured by flow cytometry. In another experiment, two doses of anti-CD38 (100 g/mice) were given intraperitoneally into these mice before intravenous injection of BK (60 mg/kg, daily) for 3 days. Forty-eight hours later mice were sacrificed and spleen, and lungs were harvested for pathology evaluation. In addition, single cell suspensions were prepared and processed for estimation of different markers (CD8, CD4, PD1, CD38) and measured by flow cytometry.

### RNA-Sequencing Analysis

CD8 T cells were treated with either IL-2 alone or activated with Ova-V or Ova peptides for 48 h. T_{frat} and T_{eff} cells were FACS-sorted and total RNA was extracted using TRIzol reagent (Invitrogen), and dissolved in RNase-free water followed by RNA-sequencing. RNA-sequencing was carried out by Maryland Genomics, Institute for Genome Sciences, UMSOM. Paired-end Illumina libraries were mapped to the Mouse reference, Ensembl release GRCm38.102, using HiSat2 v2.1.0, using default mismatch parameters. Read counts for each annotated gene were calculated using HTSeq. The DESeq2 Bioconductor package (v1.5.24) was used to estimate dispersion, normalize read counts by library size to generate the counts per million for each gene, and determine differentially expressed genes between different cell types.

RNA-sequencing analysis and pathway analysis: After filtering lowly expressed genes and normalizing counts per million using DESeq2 package the Limma package (v3.54.2) was used to perform differential gene expression on pairwise contrasts between sample conditions with an FDR<0.001 under the hierarchical testing scheme. For pathway enrichment analysis using the clusterProfiler package (v4.6.2) genes with an adjusted P value<0.001 and a log fold change greater than 1.2 were considered and evaluated against pathways retrieved from GO Biological Processes annotation set (https://geneontology.org) with a FDR<0.01. After excluding genes that were upregulated in all conditions, PD1⁺CD38^{hi} CD8⁺ T cell signature was taken as all DE upregulated genes unique to the OVAV:OVA contrast and upregulated genes OVAV:OVA∩OVAV:IL2. Proper activation signature was derived by taking upregulated genes unique to OVA:IL2 contrast and upregulated genes OVA:IL2:OVAV:IL2, since these genes were not upregulated in OVAV:OVA condition.

Single-Cell RNA-Sequencing Analysis from Patients with COVID-19

Using the publicly available single-cell RNA-sequencing data from bronchoalveolar lavage fluid (BALF) of COVID-19 patients¹⁸, sample integration and reclustering on T cells was accomplished with code provided in the publication. Subsetting CD8 T cells was accomplished by taking cells with expression of CD8A, CD8B, and CD3E greater than 0. T_{frat} cells were subset similarly with the addition of CD38 and PDCD1 genes. Plotting and subsetting functions were accomplished with the Seurat package or ggPlot2¹⁹⁻²³.

### ATAC-Sequencing Analysis

ATAC-sequencing sample preparation: Paired with the preparation of samples for the RNA-sequencing, samples were prepared for ATAC-sequencing. ATAC-sequencing analysis was carried out by Maryland Genomics, Institute for Genome Sciences, UMSOM. Paired-end Illumina libraries were mapped to the Mouse reference, Ensembl release GRCm38.102, using HiSat2 v2.1.0²⁴, using default mismatch parameters. Peak-calling was performed with MACS v2³⁵. We retained peak regions with a significant MACS p-value (FDR<0.001). Differential accessibility was performed using the DiffBind v3 R package²⁶. Differentially accessible regions with a FDR≤0.05 were used for downstream analyses. ATAC-sequencing Tn5 nick site density in T_{eff} and PD1⁺CD38^{hi} T cell promoters was analyzed to determine the relative promoter accessibility in the two cell populations. This is represented by log ratio (“minus”) vs average expression (“MA”) plot showing relative accessibility of macs2 peaks. Differential peak accessibility was indicated at an FDR<0.05. Differentially open promoters with |log 2 FC|>1.2 are illustrated by volcano plots. Row-scaled promoter accessibility for promoter-restricted peaks that are differentially regulated at an FDR<0.05, |log 2 FC|>1.2 are shown by heatmaps with pathway terms with FDR<0.05 and the genes associated with these terms depicted in the heatmap.

### Metabolic Assays

For estimation of various metabolic characteristics, FACS sorted PD1⁺CD38^{hi} CD8⁺ T cells and Teffcells were subjected to mitochondrial stress tests (SeaHorse Bioscience), performed as per the manufacturer's specifications. Oxygen consupltion rates (OCR) and extracellular acidification rates (ECAR) were measured with an XFp flux analyzer (Seahorse Bioscience). For all assays, 160,000 cells per mL were plated onto culture plates using Cell-Tak (BD Biosciences). OCR and ECAR were measured in unbuffered DMEM (Agilent Biotechnologies) supplemented with 10 mM D-glucose (Sigma-Aldrich), 10 mM L-glutamine and 2.5 mM pyruvate, as indicated. Spare respiration capacity (SRC) was calculated according to the previously published method²⁸.

### Metabolomics and Lipidomics

We used multiple reaction monitoring mass spectrometry (MRM) for the quantification of endogenous metabolites and lipids (21 classes of lipid molecules) using a triple quadrupole mass spectrometer operating in the MRM mode (QTRAP 5500 LC-MS/MS System, SCIEX). Approximately 5 million each of PD1⁺CD38^{hi} and T_{eff} cells were processed for deep metabolomics and lipidomics as we reported earlier⁸.

### Scanning Electron Microscopy (SEM)

Sorted PD1⁺CD38^{hi} and T_{eff} cells were fixed in 1% paraformaldehyde and 2.5% glutaraldehyde in 0.12 M sodium cacodylate buffer at pH 7.4. Fixed cells were embedded in 4% agarose and post-fixed in 1% osmium tetroxide (OsO₄) in 0.12 M sodium cacodylate buffer (in the dark) for 1 h. The cells were then dehydrated through graded ethanol to propylene oxide and infiltrated with 2:1, 1:1, and 1:2 propylene oxide/Epon mixtures (Embed-812; Electron Microscopy Sciences) for 90 min, 90 min, and overnight respectively, and finally, 100% Epon overnight. The beam capsules were cured at 60° C. for 48 h before sectioning. Ultrathin sections (120 nm) were cut with a Leica EM UC7 ultramicrotome (Leica Microsystems) on a diamond knife. Sections were placed in silicon wafers and carbon-taped in aluminum stubs for SEM imaging in a Helios NanoLab 660 FIBSEM (ThermoFisher). To maximize the collection of the backscattered electrons, we used a concentric detector in immersion mode at a 4 m working distance, using 2 kV and 0.10 nA current landing. High-resolution tile images of each target cell were performed using 80.000× magnification (dwell time: 5 ms, 3072×2048 resolution) with a pixel size of 1.6862 (MAPS 3.22, ThermoFisher). On the images, mitochondria were traced and counted manually and cristae morphology were examined and quantified using ImageJ V1.53a software according to the method by Lam et al²⁹.

### Estimation of NAD Levels and SIRT1 Activity

Levels of NAD in the cell lysate of FACS-sorted T_{frat} and T_{eff} cells at 0 h and 24 h after culture in IL-2 were estimated using the NAD/NADH Quantitation Kit according to the manufacturer's instructions. SIRT1 activity in these cells was measured on FACS sorted T^{frat} and T_{eff} cells by the fluorometric method (fluorescence at 360/460 nm) using the SIRT1 activity assay kit.

Quantitative PCR with Reverse Transcription Analysis

Total RNA was extracted from FACS-sorted T_{frat} and T_{eff} cells using TRIzol reagent (Invitrogen), and dissolved in RNase-free water. One g total RNA was subjected to single-strand complementary DNA synthesis using iScript cDNA Synthesis Kit (Bio-Rad). The Taqman® gene expression assay from Applied Biosystems was used for qrtPCR, and primer pairs (Catalog no. 4331182) from Applied Biosystems were used to detect gene expression of Foxo1 (Mm00490671_ml) and Tcf-7 (Mm00493445_ml). Expression data were procured using StepOnePlus Real-Time PCR System from Applied Biosystems and normalized to the geometric mean of the Applied Biosystems™ Mouse Actb (actin, beta) Endogenous Control (cat No 4352933E).

### Western Blot

FACS-sorted T^{frat} and T_{eff} cells were activated as described above. In addition, primary murine CD8⁺ T cells, as well as human CD8⁺ T cells were isolated and activated as described above and in some group various concentrations (murine: 50 nM-1000 nM; human: 3-15 μM) of recombinant-BK were added for 48 hours. Cell lysates (RIPA buffer+1% phosphatase inhibitor+1% protease inhibitor) were prepared after various treatments. Protein concentrations in cell lysates were determined by Pierce BCA Protein Assay Kit (ThermoFisher Scientific). Protein (30-40 μg) was loaded onto Novex 4-12% Tris-Glycin Mini Gels (ThermoFisher Scientific) followed by transfer onto nitrocellulose membranes. Membranes were blocked with 3% BSA in Tris buffer followed by overnight probing with antibodies against SIRT1, Foxo1, TCF-1, phospho-CREB (Ser133) rabbit mAb (Catalog #9198), CREB (catalog No. 9197S) by Western blot. The blots were developed with appropriate (anti-rabbit or anti-mouse) HRP-conjugated secondary antibodies. The level of tubulin and Lamin B were used as controls. Densitometric analysis of the bands was performed using a software (Image Studio Lite v.5.0) from LI-COR (https://www.licor.com/bio/image-studio-lite/).

### IF Microscopy

FACS-sorted PD1⁺CD38^{hi} CD8 T cells generated as above were treated with DAPI and Gzm B specific stain as per the manufacturer's instructions. After washing free stains, stained killer T^{frat} cells were mixed with non-stained target pMel-1 CD8 T cells at a ratio of 5:1 (killer:target) and placed on CellTak coated (22.5 g/ml) slides. Transfer of Gzm B into target cells was followed in a time dependent manner (5-300 minutes) using Leica microscope (LEICA SP8).

### Statistical Analysis

All summary statistics (average values, s.d., s.e.m., significant differences between groups) were calculated using GraphPad Prism v.6.0 or Excel, as appropriate. Statistical significance between groups was determined by unpaired, one-tailed Student's t-test or one-way analysis of variance (ANOVA) (P<0.05 was considered statistically significant). Survival in various groups was compared with GraphPad Prism using log-rank (Mantel-Cox) test.

### REFERENCES

1 Huang, A. C. et al. T-cell invigoration to tumour burden ratio associated with anti-PD-1 response. Nature 545, 60-65, doi:10.1038/nature22079 (2017).
2 Ji, Y. et al. Identification of the genomic insertion site of Pmel-1 TCR alpha and beta transgenes by next-generation sequencing. PLoS One 9, e96650, doi:10.1371/journal.pone.0096650 (2014).
3 Morel, L. et al. Functional dissection of systemic lupus erythematosus using congenic mouse strains. J Immunol 158, 6019-6028 (1997).
4 Hemmi, H. et al. A Toll-like receptor recognizes bacterial DNA. Nature 408, 740-745, doi:10.1038/35047123 (2000).
5 Hemmi, H. et al. Small anti-viral compounds activate immune cells via the TLR7 MyD88-dependent signaling pathway. Nat Immunol 3, 196-200, doi:10.1038/ni758 (2002).
6 Hwang, S. H. et al. B cell TLR7 expression drives anti-RNA autoantibody production and exacerbates disease in systemic lupus erythematosus-prone mice. J Immunol 189, 5786-5796, doi:10.4049/jimmunol.1202195 (2012).
7 Verma, V. et al. PD-1 blockade in subprimed CD8 cells induces dysfunctional PD-1(+)CD38(hi) cells and anti-PD-1 resistance. Nat Immunol 20, 1231-1243, doi:10.1038/s41590-019-0441-y (2019).
8 Verma, V. et al. MEK inhibition reprograms CD8(+) T lymphocytes into memory stem cells with potent antitumor effects. Nat Immunol 22, 53-66, doi:10.1038/s41590-020-00818-9 (2021).
9 Miller, S. D. & Karpus, W. J. Experimental autoimmune encephalomyelitis in the mouse. Current protocols in immunology Chapter 15, 15.11.11-15.11.18, doi:10.1002/0471142735.im1501s77 (2007).
Bittner, S., Afzali, A. M., Wiendl, H. & Meuth, S. G. Myelin oligodendrocyte glycoprotein (MOG35-55) induced experimental autoimmune encephalomyelitis (EAE) in C57BL/6 mice. J Vis Exp, doi:10.3791/51275 (2014).
11 Barthelmes, J. et al. Induction of Experimental Autoimmune Encephalomyelitis in Mice and Evaluation of the Disease-dependent Distribution of Immune Cells in Various Tissues. J Vis Exp, doi:10.3791/53933 (2016).
12 Fairhurst, A. M. et al. Systemic IFN-alpha drives kidney nephritis in B6.Sle123 mice. Eur J Immunol 38, 1948-1960, doi:10.1002/eji.200837925 (2008).
13 Lin, K. Y. et al. Treatment of established tumors with a novel vaccine that enhances major histocompatibility class II presentation of tumor antigen. Cancer Res 56, 21-26 (1996).
14 Shrimali, R. K. et al. Concurrent PD-1 Blockade Negates the Effects of OX40 Agonist Antibody in Combination Immunotherapy through Inducing T-cell Apoptosis. Cancer Immunol Res 5, 755-766, doi:10.1158/2326-6066.CIR-17-0292 (2017).
15 Sukumar, M. et al. Mitochondrial Membrane Potential Identifies Cells with Enhanced Sternness for Cellular Therapy. Cell Metab 23, 63-76, doi:10.1016/j.cmet.2015.11.002 (2016).
16 Su, Y. et al. Multi-Omics Resolves a Sharp Disease-State Shift between Mild and Moderate COVID-19. Cell 183, 1479-1495 e1420, doi:10.1016/j.cell.2020.10.037 (2020).
17 Su, Y. et al. Multiple early factors anticipate post-acute COVID-19 sequelae. Cell 185, 881-895 e820, doi:10.1016/j.cell.2022.01.014 (2022).
18 Liao, M. et al. Single-cell landscape of bronchoalveolar immune cells in patients with COVID-19. Nature Medicine 26, 842-844, doi:10.1038/s41591-020-0901-9 (2020).
19 Hao, Y. et al. Integrated analysis of multimodal single-cell data. Cell 184, 3573-3587 e3529, doi:10.1016/j.cell.2021.04.048 (2021).
20 Stuart, T. et al. Comprehensive Integration of Single-Cell Data. Cell 177, 1888-1902 e1821, doi:10.1016/j.cell.2019.05.031 (2019).
21 Butler, A., Hoffman, P., Smibert, P., Papalexi, E. & Satija, R. Integrating single-cell transcriptomic data across different conditions, technologies, and species. Nat Biotechnol 36, 411-420, doi:10.1038/nbt.4096 (2018).
22 Satija, R., Farrell, J. A., Gennert, D., Schier, A. F. & Regev, A. Spatial reconstruction of single-cell gene expression data. Nat Biotechnol 33, 495-502, doi:10.1038/nbt.3192 (2015).
23 Wickham, H. ggplot2: Elegant Graphics for Data Analysis. 2 edn, (Springer Cham, 2016).
24 Kim, D., Paggi, J. M., Park, C., Bennett, C. & Salzberg, S. L. Graph-based genome alignment and genotyping with HISAT2 and HISAT-genotype. Nat Biotechnol 37, 907-915, doi:10.1038/s41587-019-0201-4 (2019).
25 Zhang, Y. et al. Model-based analysis of ChIP-Seq (MACS). Genome Biol 9, R137, doi:10.1186/gb-2008-9-9-r137 (2008).
26 Ross-Innes, C. S. et al. Differential oestrogen receptor binding is associated with clinical outcome in breast cancer. Nature 481, 389-393, doi:10.1038/nature10730 (2012).
27 Heinz, S. et al. Simple combinations of lineage-determining transcription factors prime cis-regulatory elements required for macrophage and B cell identities. Mol Cell 38, 576-589, doi:10.1016/j.molcel.2010.05.004 (2010).
28 van der Windt, G. J. et al. Mitochondrial respiratory capacity is a critical regulator of CD8⁺ T cell memory development. Immunity 36, 68-78, doi:10.1016/j.immuni.2011.12.007
   S1074-7613(11)00518-8 [pii](2012).
29 Lam, J. et al. A Universal Approach to Analyzing Transmission Electron Microscopy with ImageJ. Cells 10, doi:10.3390/cells10092177 (2021).

1. A method for decreasing apoptosis of cells in a subject comprising decreasing binding of a CD38-expressing cell to a CD31-expressing cell in the subject.

2. The method of claim 1, wherein binding is decreased by administering an agent that decreases binding of a CD38-expressing cell to a CD31-expressing cell in the subject.

3. The method of claim 1, wherein binding is decreased by decreasing the number of PD-1⁺CD38^{hi}CD8⁺ T cells in the subject.

4. The method of claim 3, wherein the number of PD-1⁺CD38^{hi}CD8⁺ T cells is decreased by:
(a) reducing the number of PD-1⁺CD38^{hi}CD8⁺ T cells ex vivo in a biological sample from the subject; and
(b) administering the remaining population of cells to the subject.

5. The method of claim 1, wherein the CD31-expressing cell is a white blood cell or an endothelial cell.

6. The method of claim 5, wherein the CD31-expressing white blood cell is selected from the group consisting of a T lymphocyte, a dendritic cell, a natural killer cell, and a macrophage.

7. The method of claim 6, wherein the CD31-expressing white blood cell is a T lymphocyte.

8. The method of claim 7, wherein the T lymphocyte is a CD4⁺ T cell or a CD8+ T cell.

9. The method of claim 1, wherein the CD38-expressing cell is a CD8⁺ T cell.

10. The method of claim 9, wherein the CD8⁺ T cell is a PD-1⁺CD38^{hi}CD8⁺ T cell.

11. The method of claim 2, wherein the agent is an antibody that specifically binds to CD31.

12. The method of claim 2, wherein the agent is an antibody that specifically binds to CD38.

13. The method of claim 2, wherein the agent reduces the level of PD-1⁺CD38^{hi}CD8⁺ T cells in the subject.

14. The method of claim 1, wherein degranulation of Granzyme B (GzmB) in PD-1⁺CD38^{hi}CD8⁺ T cells is reduced in the subject.

15. The method of claim 14, wherein transfer of GzmB from PD-1⁺CD38^{hi}CD8⁺ T cells into white blood cells in the subject is reduced.

16. The method of claim 1, wherein the subject has acute respiratory distress syndrome (ARDS).

17. The method of claim 16, wherein the subject has an infection associated with ARDS.

18. The method of claim 17, wherein the subject has COVID-19.

19. A method of treating an autoimmune disease in a subject comprising administering to the subject a population of PD-1⁺CD38^{hi}CD8⁺ T cells.

20. The method of claim 19, further comprising producing the population of PD-1⁺CD38^{hi}CD8⁺ T cells prior to administration to the subject.

21. The method of claim 20, wherein the CD8⁺ T cells are produced by contacting CD8⁺ T cells with a suboptimal antigen and/or a PD-1 inhibitor prior to administering the population of PD-1⁺CD38^{hi}CD8⁺ T cells to the subject.

22. The method of claim 21, wherein the PD-1 inhibitor is an anti-PD-1 antibody or an anti-PD-L1 antibody.

23. The method of claim 20, wherein the population of PD-1⁺CD38^{hi}CD8⁺ T cells is expanded prior to administration to the subject.

24. The method of claim 19, wherein the CD8⁺ T cells are autologous CD8⁺ T cells.

25. The method of claim 19, wherein the CD8⁺ T cells are homologous CD8⁺ T cells.

26. The method of claim 1, further comprising administering a second therapeutic agent to the subject.

27. The method of claim 26, wherein the second therapeutic agent is an immunomodulator.

28. The method of claim 27, wherein the immunomodulator is an immunosuppressant.

29. The method of claim 27, wherein the immunomodulator is an immunostimulant.


SEQUENCE LISTING (4 sequences)

SEQ ID NO: 1 - protein, 8 aa, synthetic construct
SIINFEKL

SEQ ID NO: 2 - protein, 8 aa, synthetic construct
SIIGFEKL

SEQ ID NO: 3 - protein, 17 aa, synthetic construct
ISQAVHAAHAEINEAGR

SEQ ID NO: 4 - protein, 9 aa, synthetic construct
KVPRNQDWL